(19) United States
(12) Patent Application Publication
(10) Pub. No.: US 2022/0378954 A1
(43) Pub. Date: Dec. 1, 2022

(54) METHODS FOR RADIOLABELLING GRPR ANTAGONISTS AND THEIR KITS

(71) Applicant: NOVARTIS AG, Basel (CH)

(72) Inventors: Elena CASTALDI, Ivrea (IT); Lorenza FUGAZZA, Ivrea (IT); Maurizio F. MARIANI, Ivrea (IT); Francesca ORLANDI, Ivrea (IT); Mattia TEDESCO, Ivrea (IT)

(21) Appl. No.: 17/753,850

(22) PCT Filed: Sep. 16, 2020
(86) PCT No.: PCT/EP2020/075911

§ 371 Date: Mar. 16, 2022

(30) Foreign Application Priority Data
Sep. 17, 2019 (EP) 19197833.7
Apr. 29, 2020 (EP) 20172142.0

(51) Int. Cl. A61K 51/08; C07K 1/13; A61K 51/12; A61K 47/12; A61K 9/00

(52) U.S. Cl. CPC A61K 51/088; C07K 1/13; A61K 51/121; A61K 47/12; A61K 9/0019; A61K 51/1286

(57) ABSTRACT

The present invention relates to methods for radiolabelling GRPR antagonists such as NeoB, and their kits. In particular, the invention to a method for labeling a gastrin-releasing peptide receptor (GRPR) antagonist with a radioactive isotope, preferably ⁶⁸Ga, ⁶⁷Ga or ⁶⁴Cu, said method comprising the steps of: i. providing a first vial comprising said GRPR antagonist in dried form, ii. adding a solution of said radioactive isotope into said first vial, thereby obtaining a solution of said GRPR antagonist with said radioactive isotope, iii. mixing the solution obtained in ii. with at least a buffering agent and incubating it for a sufficient period of time for obtaining said GRPR antagonist labeled with said radioactive isotope, and, iv. optionally, adjusting the pH of the solution.

### TECHNICAL FIELD

The present disclosure relates to methods for radiolabelling GRPR antagonists such as NeoB, and their kits.

### BACKGROUND

Bombesin was first isolated from the European frog Bombina bombina and was demonstrated to mimic the mammalian gastrin-releasing peptide (GRP) and neuromedin B (NMB): Erspamer, V. Discovery, Isolation, and Characterization of Bombesin-like Peptides. Ann N Y Acad Sci 547: 3-9, 1988; Jensen, R. T.; Battey, J. F.; Spindel, E. R.; Benya, R. V. International union of pharmacology. LXVIII. Mammalian bombesin receptors: Nomenclature, distribution, pharmacology, signaling, and functions in normal and disease states. Pharmacol. Rev. 2008, 60, 1-42].

Gastrin-releasing peptide (GRP), a bombesin-like peptide growth factor, regulates numerous functions of the gastrointestinal and central nervous systems, including release of gastrointestinal hormones, smooth muscle cell contraction, and epithelial cell proliferation. It is a potent mitogen for physiologic and neoplastic tissues, and it may be involved in growth dysregulation and carcinogenesis.

The effects of GRP are primarily mediated through binding to its receptor, the GRP receptor (GRPR), a G protein—coupled receptor originally isolated from a small cell lung cancer cell line. Upregulation of the pathway of GRP/GRPR has been reported in several cancers, including breast, prostate, uterus, ovaries, colon, pancreas, stomach, lung (small and non-small cell), head and neck squamous cell cancer and in various cerebral and neural tumours.

GRPR are highly overexpressed in prostate cancer where studies in human prostate cancer cell-lines and xenograft models showed both high affinity (nM level) and high tumour uptake (% ID/g) but the relative expression of GRPR across evolving disease setting from early to late stage has not been fully elucidated yet [Waters, et al. 2003, Br J Cancer. June 2; 88(11): 1808-1816].

In colorectal patients, presence of GRP and expression of GRPR have been determined by immunohistochemistry in randomly selected colon cancers samples, including LN and metastatic lesions. Over 80% of samples aberrantly expressed either GRP or GRPR, and over 60% expressing both GRP and GRPR, whereas expression was not observed in adjacent normal healthy epithelium [Scopinaro F, et al. Cancer Biother Radiopharm 2002, 17(3):327-335].

GRP is physiologically present in pulmonary neuroendocrine cells and plays a role in stimulating lung development and maturation. However, it seems to also be involved in growth dysregulation and carcinogenesis. Stimulation of GRP leads to increasing the release of epidermal growth factor receptor (EGFR) ligands with subsequent activation of EGFR and mitogen-activated protein kinase downstream pathways. Using non-small cell lung cancer (NSCLC) cell lines it has been confirmed that EGF and GRP both stimulate NSCLC proliferation, and inhibition of either EGFR or GRPR resulted in cell death [Shariati F, et al. Nucl Med Commun 2014, 35(6):620-625].

In nuclear medicine, peptide receptor agonists have long been the ligands of choice for tracer development and utilization. The rationale behind the use of agonist-based constructs laid on to receptor-radioligand complex internalization enabling the high accumulation of radioactivity inside the target cells. In case of radiometal-labelled peptides, the efficient receptor-mediated endocytosis in response to agonist stimulation provides high in vivo radioactivity uptake in targeted tissues, a crucial prerequisite for optimal imaging of malignancies. However, a paradigm shift occurred when receptor-selective peptide antagonists showed preferable biodistribution, including considerably greater in vivo tumour uptake, compared with highly potent agonists. A further advantage displayed by GRPR antagonists is a safer clinical use, not so much at tracer doses for the current diagnostic point of view, but in view of greater doses for potential therapeutic purposes, as the use of antagonists does not foresee acute biological adverse effects [Stoykow C, et al. Theragnostics 2016, 6(10):1641-1650].

In non-clinical models, [₆₈Ga]-NeoB and [₁₇₇Lu]-NeoB (also called [₆₈Ga]-NeoBOMB1 and [₁₇₇Lu]-NeoBOMB1) have shown high affinity to the GRPR expressed in breast, prostate, and Gastro Intestinal Stromal Tumor (GIST), as well as a low degree of internalization upon binding to the specific receptor. The ability of the radiolabeled peptide to target the GRPR expressing tumor has been confirmed in in vivo imaging and biodistribution studies in animal models [Dalm et al Journal of nuclear medicine 2017, Vol. 58(2): 293-299; Kaloudi et al. Molecules, 2017 Nov. 11;22(11); Paulmichl A et al. Cancer Biother Radiopharm, 2016 Oct.; 31(8):302-310].

However, no optimized method has been developed for labeling NeoB with ⁶⁸Ga, ⁶⁷Ga or ⁶⁴Cu to thereby obtain labeled NeoB solution for imaging purposes of GRPR-positive tumors in human patients. In particular, there is need for a rapid, efficient and safe procedure which would provide a high radiochemical purity of labeled GRPR antagonist, such as [⁶⁸Ga] NeoB for intravenous injection in human subject in need thereof.

### SUMMARY

One first aspect of the disclosure relates to a method for labeling a gastrin-releasing peptide receptor (GRPR) antagonist with a radioactive isotope, preferably ⁶⁸Ga, ⁶⁷Ga or ⁶⁴Cu, said method comprising the steps of:
i. providing a first vial comprising said GRPR antagonist in dried form,
ii. adding a solution of said radioactive isotope into said first vial, thereby obtaining a solution of said GRPR antagonist with said radioactive isotope,
iii. mixing the solution obtained in ii. with at least a buffering agent and incubating it for a sufficient period of time for obtaining said GRPR antagonist labeled with said radioactive isotope, and,
iv. optionally, adjusting the pH of the solution.

In specific embodiments, said radioactive isotope is ⁶⁸Ga and the radiochemical purity as measured in HPLC is at least 90%, and optionally, the percentage of free ⁶⁸Ga3+ (in HPLC) is 2% or less, and/or the percentage of non-complexed ⁶⁸Ga3+ species (in ITLC) is 5% or less.

In other specific embodiments, said radioactive isotope is ⁶⁷Ga and the radiochemical purity as measured in HPLC is at least 90%, and optionally, the percentage of free ⁶⁷Ga3+ (in HPLC) is 2% or less, and/or the percentage of non-complexed ⁶⁷Ga3+ species (in ITLC) is 5% or less.

In other specific embodiments, said radioactive isotope is ⁶⁴Cu and the radiochemical purity as measured in HPLC is at least 90%, and optionally, the percentage of free ⁶⁴Cu2+ (in HPLC) is 2% or less, and/or the percentage of non-complexed ⁶⁴Cu2+ species (in ITLC) is 5% or less.

Preferably, the GRPR antagonist is NeoB compound of formula (I):

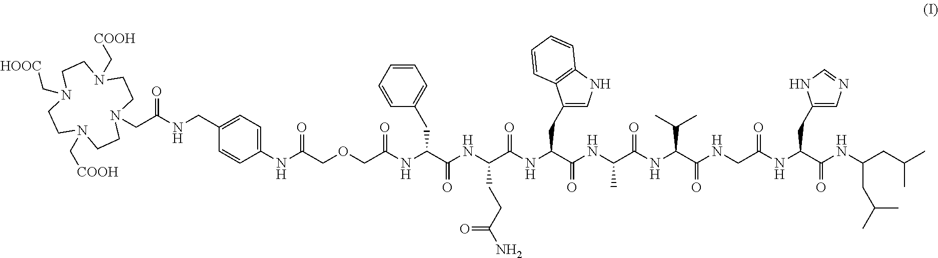 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

(DOTA-(p-aminobenzylamine-diglycolic acid))-[D-Phe-Gln-Trp-Ala-Val-Gly-His-NH—CH[CH₂—CH(CH₃)₂]₂

In another aspect, the disclosure relates to a solution comprising a GRPR antagonist labeled with a radioactive isotope, obtainable or obtained by the methods as disclosed herein, for use as an injectable solution for in vivo detection of tumors by imaging in a subject in need thereof.

It is another object of the present disclosure to provide a powder for solution for injection, comprising the following components in dried forms:
i. A GRPR antagonist of the following formula:

C—S—P
wherein:
C is a chelator capable of chelating said radioactive isotope;
S is an optional spacer covalently linked between C and the N-terminal of P;
P is a GRPR peptide antagonist, preferably of the general formula:

Xaa1-Xaa2-Xaa3-Xaa4-Xaa5-Xaa6-Xaa7-Z;
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) (all as L- or D-isomers);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl)histidine (3-Me)His;
Z is selected from —NHOH, —NHNH₂, —NH-alkyl, —N(alkyl)₂, and —O-alkyl or Z is

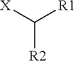

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido, or amide substituted aryl or heteroaryl group;
ii. a radiolysis protector, for example gentisic acid;
iii. a bulking agent, for example, mannitol; and,
iv. optionally a surfactant, for example macrogol 15 hydroxystearate.

Typically, said powder for solution for injection comprises the following components:
i. NeoB of the following formula (I) at an amount between 20 and 60 μg, typically, 50 μg;

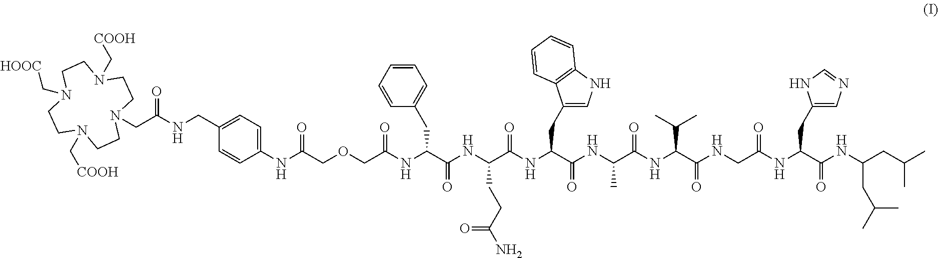 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

ii. gentisic acid at an amount of 50 and 250 μg, typically, 200 μg, and,
iii. mannitol at an amount between 10 and 30 mg, for example 20 mg, and,
iv. macrogol 15 hydroxystearate at an amount between 250 and 750 μg, for example 500 μg.

The present disclosure further relates to a kit for carrying out the above labeling method, comprising
i. a first vial with the following components in dried forms
   i. NeoB of the following formula (I):

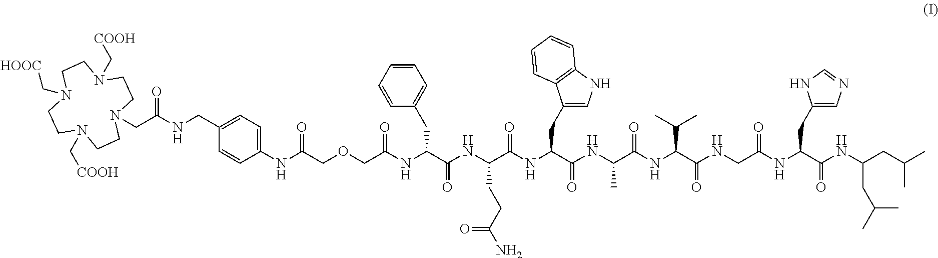 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

ii. a radiolysis protector, for example gentisic acid,
iii. optionally a bulking agent, for example, mannitol, and,
iv. optionally a surfactant, for example macrogol 15 hydroxystearate; and,
ii. a second vial comprising at least a buffering agent, preferably in dried form; and,
iii. optionally, an accessory cartridge for eluting a radioactive isotope generated by a radioactive isotope generator.

Another kit herein disclosed comprises a single vial with the following components in dried forms
i. NeoB of the following formula (I):

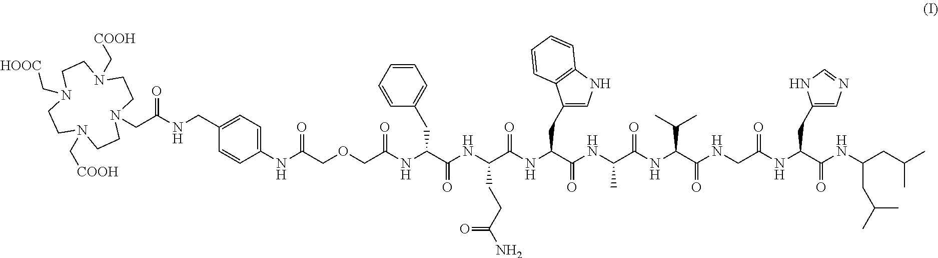 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

i. a radiolysis protector, for example gentisic acid,
ii. optionally a bulking agent, for example, mannitol,
iii. optionally a surfactant, for example macrogol 15 hydroxystearate, and,
iv. at least a buffering agent, preferably in dried form; and,
ii. optionally, an accessory cartridge for eluting a radioactive isotope generated by a radioactive isotope generator.

For example, the kit may comprise a first or single vial with the following components:
i. NeoB of the following formula (I) at an amount between 20 and 60 μg, typically, 50 μg;

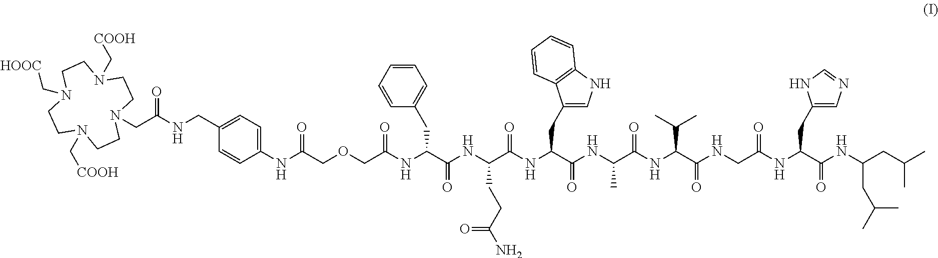 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

ii. gentisic acid at an amount of 50 and 250 μg, typically, 200 μg,
iii. mannitol at an amount between 10 and 30 mg, for example 20 mg, and,
iv. optionally, macrogol 15 hydroxystearate at an amount between 250 and 750 μg, for example 500 g.

### DETAILED DESCRIPTION OF THE INVENTION

In general, the present disclosure relates to a method for labeling a gastrin-releasing peptide receptor (GRPR) antagonist with a radioactive isotope, preferably ⁶⁸Ga, ⁶⁷Ga or ⁶Cu, said method comprising the steps of:
(i) providing a first vial comprising said GRPR antagonist in dried form,
(ii) adding a solution of said radioactive isotope into said first vial, thereby obtaining a solution of said GRPR antagonist with said radioactive isotope,
(iii) mixing the solution obtained in ii. with at least a buffering agent and incubating it for a sufficient period of time for obtaining said GRPR antagonist labeled with said radioactive isotope, and,
(iv) optionally, adjusting the pH of the solution.

The radiolabeled GRPR antagonist obtained by the disclosed methods is preferably a radioactive GRPR antagonist for use as a contrast agent for PET/CT, SPECT or PET/MRI imaging.

A preferred radiolabeled GRPR antagonist obtained by the disclosed methods is the NeoB compound labelled with a radioactive isotope suitable for use as a contrast agent for PET/CT, SPECT or PET/MRI imaging, preferably ⁶⁸Ga, ⁶⁷Ga or ⁶⁴Cu. In a preferred embodiment, ⁶⁷Ga is used for SPECT imaging and ⁶⁸Ga and ⁶⁴Cu are used for PET imaging such as PET/CT or PET/MRI.

The methods of the present disclosure may advantageously provide excellent radiochemical purity of the radiolabelled compound, e.g. radiolabeled NeoB compound with ⁶⁸Ga, typically the radiochemical purity as measured in HPLC is at least 92%, and optionally, the percentage of free ⁶⁸Ga3+ (in HPLC) is 2% or less, and/or the percentage of non-complexed ⁶⁸Ga3+ species (in ITLC) is 3% or less.

Assays for measuring radiochemical purity in HPLC or in ITLC and free ⁶⁸Ga3+ are further described in detail in the Examples.

### Definitions

The phrase “treatment of” and “treating” includes the amelioration or cessation of a disease, disorder, or a symptom thereof. In particular, with reference to the treatment of a tumor, the term “treatment” may refer to the inhibition of the growth of the tumor, or the reduction of the size of the tumor.

Consistent with the International System of Units, “MBq” is the abbreviation for the unit of radioactivity “megabecquerel.”

As used herein, “PET” stands for positron-emission tomography.

As used herein, “SPECT” stands for single-photon emission computed tomography.

As used herein, “MRI” stands for magnetic resonance imaging.

As used herein, “CT” stands for computed tomography.

As used herein, the terms “effective amount” or “therapeutically efficient amount” of a compound refer to an amount of the compound that will elicit the biological or medical response of a subject, for example, ameliorate the symptoms, alleviate conditions, slow or delay disease progression, or prevent a disease.

As used herein, the terms “substituted” or “optionally substituted” refers to a group which is optionally substituted with one or more substituents selected from: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″ —S(O)R′, —S(O)₂R′, —S(O)₂NR′R″, —NRSO₂R′, —CN, —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, alkyl, heteroalkyl, cycloalkyl, heterocycloalkyl, aryl and heteroaryl. When a compound of the disclosure includes more than one R group, for example, each of the R groups is independently selected as are each R′, R″, R′″ and R″″ groups when more than one of these groups is present.

As used herein, the terms “alkyl”, by itself or as part of another substituent, refer to a linear or branched alkyl functional group having 1 to 12 carbon atoms. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

As used herein, the terms “heteroaryl” refer to a polyunsaturated, aromatic ring system having a single ring or multiple aromatic rings fused together or linked covalently, containing 5 to 10 atoms, wherein at least one ring is aromatic and at least one ring atom is a heteroatom selected from N, O and S. The nitrogen and sulfur heteroatoms may optionally be oxidized and the nitrogen heteroatoms may optionally be quaternized. Such rings may be fused to an aryl, cycloalkyl or heterocyclyl ring. Non-limiting examples of such heteroaryl, include: furanyl, thiophenyl, pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzothiadiazolyl, quinolinyl, isoquinolinyl, cinnolinyl, quinazolinyl and quinoxalinyl.

As used herein, the terms “aryl” refer to a polyunsaturated, aromatic hydrocarbyl group having a single ring or multiple aromatic rings fused together, containing 6 to 10 ring atoms, wherein at least one ring is aromatic. The aromatic ring may optionally include one to two additional rings (cycloalkyl, heterocyclyl or heteroaryl as defined herein) fused thereto. Suitable aryl groups include phenyl, naphtyl and phenyl ring fused to a heterocyclyl, like benzopyranyl, benzodioxolyl, benzodioxanyl and the like.

As used herein, the term “halogen” refers to a fluoro (—F), chloro (—Cl), bromo (—Br), or iodo (—I) group

As used herein the terms “optionally substituted aliphatic chain” refers to an optionally substituted aliphatic chain having 4 to 36 carbon atoms, preferably 12 to 24 carbon atoms.

As used herein, the term “chelator” refers to a molecule with functional groups such as amines or carboxylic group suitable to complex the radioactive isotope via non-covalent bonds.

As used herein, the term «radiolysis protector» refers to a stabilizing agent which protects organic molecules against radiolytic degradation, e.g. when a gamma ray emitted from the radionuclide is cleaving a bond between the atoms of an organic molecules and radicals are formed, those radicals are then scavenged by the stabilzer which avoids the radicals undergoing any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”.

As used herein, the term “Radiochemical purity” refers to that percentage of the stated radionuclide that is present in the stated chemical or biological form. Radiochromatography methods, such as HPLC method or instant Thin Layer Chromatography method (iTLC), are the most commonly accepted methods for determining radiochemical purity in the nuclear pharmacy.

If not stated herein otherwise, “about” means ±20%, preferably ±10%, more preferably ±5%, even more preferably ±2%, even more preferably ±1%. The term “about” is herein used synonymous with “ca.”

Step (i) of Providing a First Vial Comprising Said GRPR Antagonist in Dried Form

The GRPR Antagonist

As used herein, said GRPR antagonist has the following formula:

C—S—P

wherein:
C is a chelator capable of chelating the radioactive isotope;
S is an optional spacer covalently linked between C and the N-terminal of P;
P is a GRPR peptide antagonist, preferably of the general formula:

Xaa1-Xaa2-Xaa3-Xaa4Xaa5-Xaa6-Xaa7-Z;
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) (all as L- or D-isomers);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl)histidine (3-Me)His;
Z is selected from —NHOH, —NHNH2, —NH-alkyl, —N(alkyl)2, and —O-alkyl or Z is

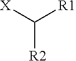

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido, or amide substituted aryl or heteroaryl group.

According to an embodiment, Z is selected from one of the following formulae, wherein X is NH or O:

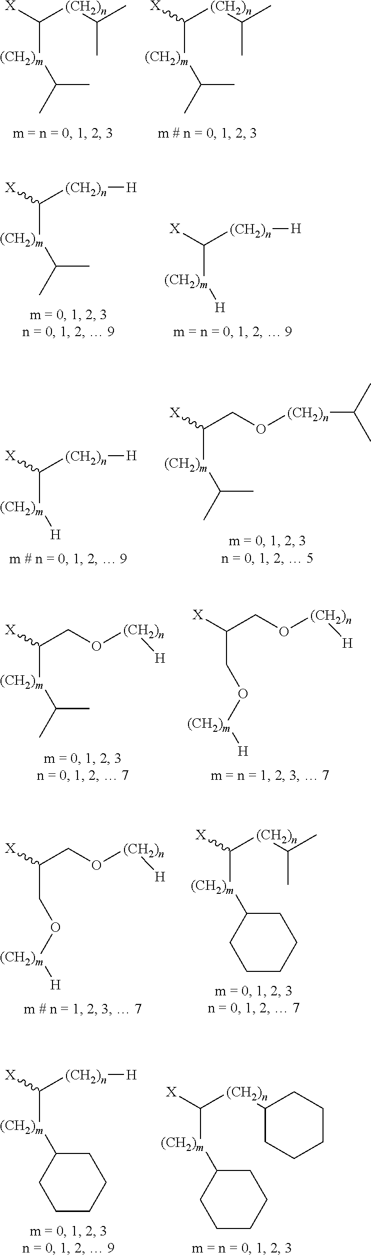

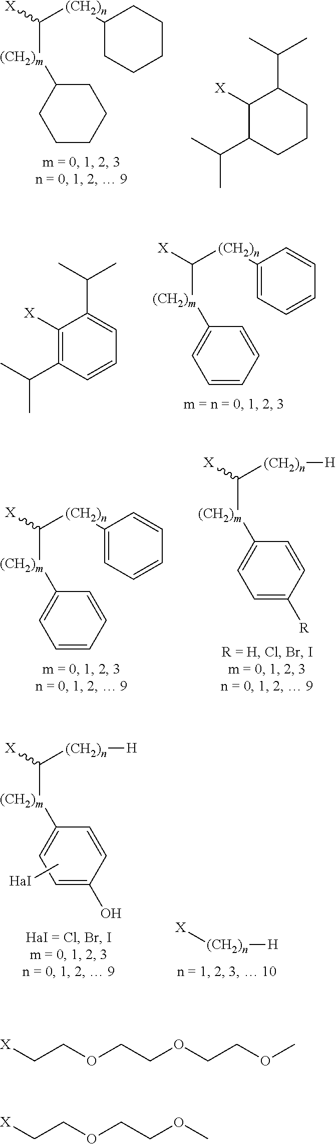

According to an embodiment, P is DPhe-Gln-Trp-Ala-Val-Gly-His-Z; wherein Z is defined as above.

According to an embodiment, P is DPhe-Gln-Trp-Ala-Val-Gly-His-Z;

Z is selected from Leu-ψ(CH2N)-Pro-NH2 and NH—CH(CH₂—CH(CH₃)₂)₂

or Z is

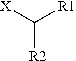

wherein X is NH (amide) and R2 is CH(CH₂—CH(CH₃)₂ and R1 is the same as R2 or different (CH2N)-Pro-NH2.

According to an embodiment, the chelator C is obtained by grafting one chelating agent selected among the following list:

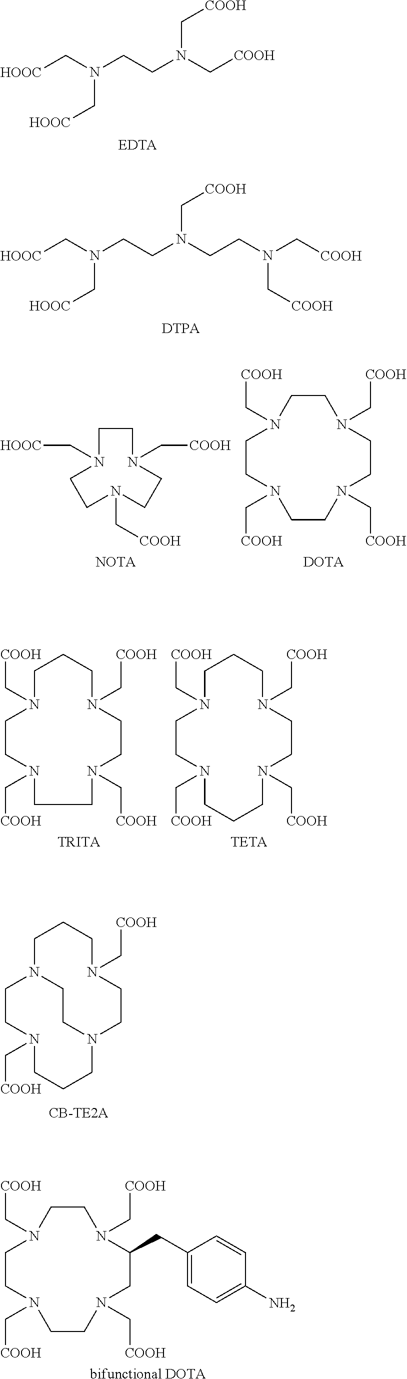

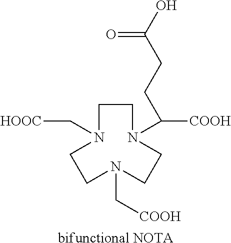 [SMILES: O=C(O)CCC(C(=O)O)N1CCN(CC(=O)O)CCN(CC(=O)O)CC1 ; "bifunctional NOTA"]

In specific embodiments, C is obtained by grafting a chelating agent selected from the group consisting of:

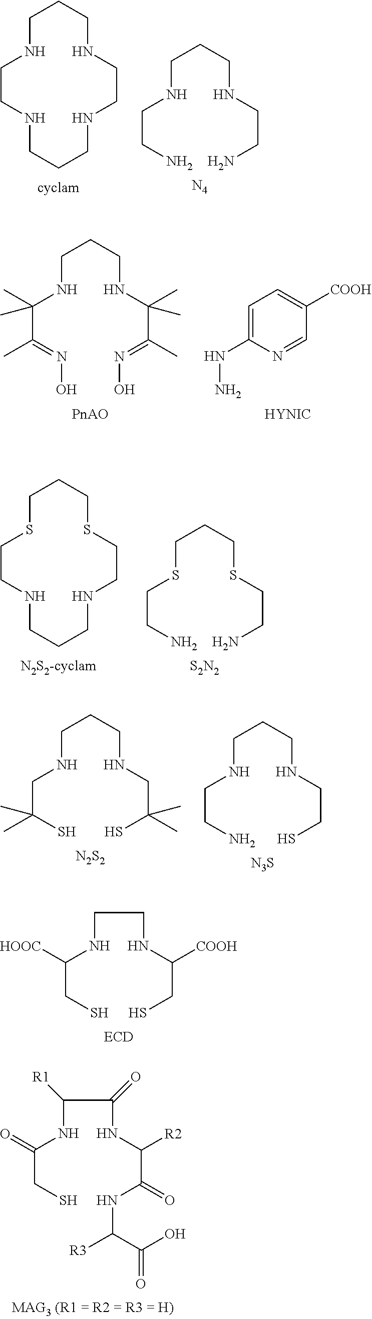

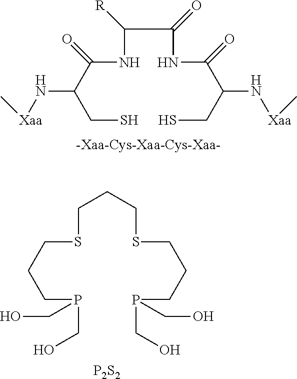

According to an embodiment, S is selected from the group consisting of:

a) aryl containing residues of the formulae:

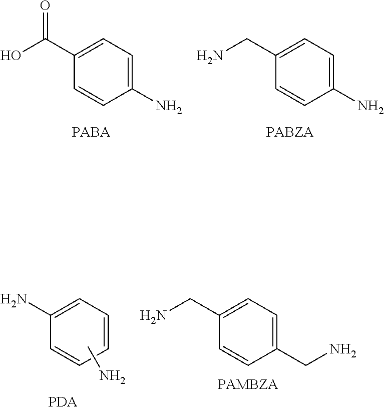

wherein PABA is p-aminobenzoic acid, PABZA is p-aminobenzylamine, PDA is phenylenediamine and PAMBZA is (aminomethyl) benzylamine;

b) dicarboxylic acids, ω-aminocarboxylic acids,ω-diaminocarboxylic acids or diamines of the formulae:

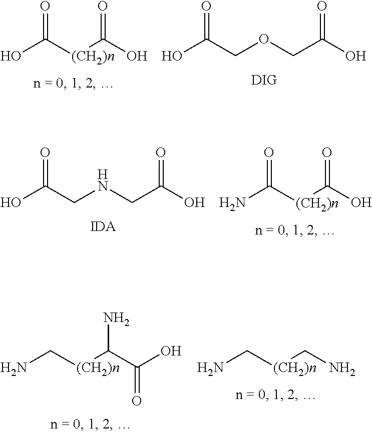

wherein DIG is diglycolic acid and IDA is iminodiacetic acid;

c) PEG spacers of various chain lengths, in particular PEG spacers selected from the following:

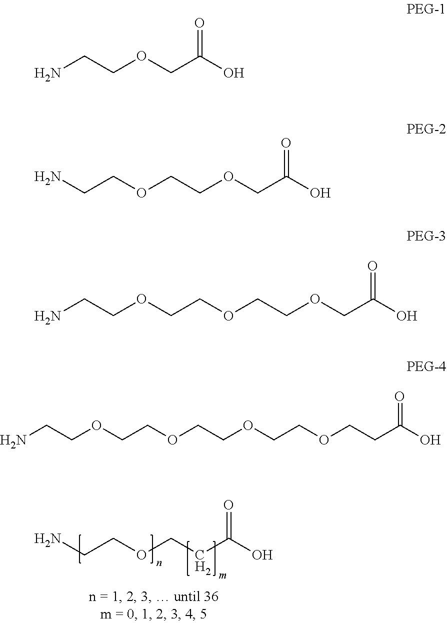

d) α- and β-amino acids, single or in homologous chains various chain lengths or heterologous chains of various chain lengths, in particular:

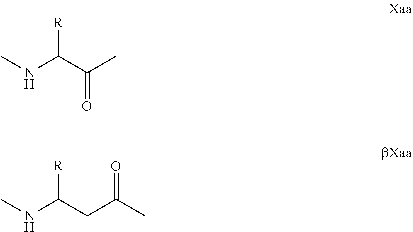

GRP(1-18), GRP(14-18), GRP(13-18), BBN(1-5), or [Tyr4] BB (1-5); or

e) combinations of a, b, c and d.

According to a particular embodiment, the radiolabelled GRPR antagonist is selected from the group consisting of compounds of the following formulae:

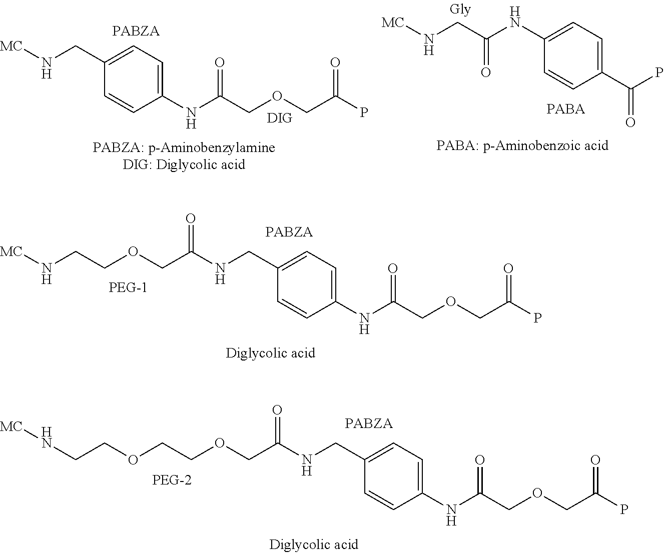

wherein C and P are as defined above, and M is the radioactive isotope, preferably M is selected from ⁶⁸Ga, ⁶⁷Ga or ⁶⁴Cu.

According to a preferred embodiment, the GRPR-antagonist is NeoB (also called NeoBOMB1) of formula (I):

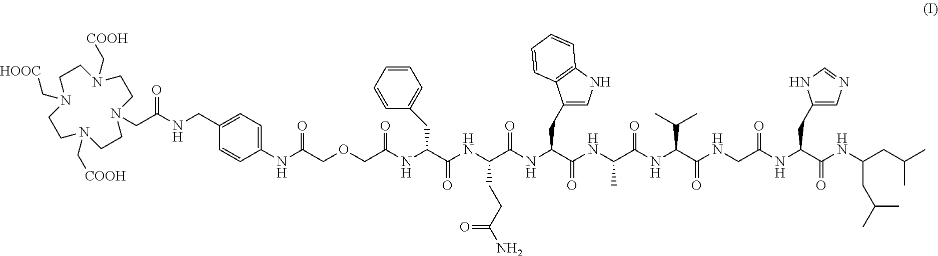 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

(DOTA-(p-aminobenzylamine-diglycolic acid))-[D-Phe-Gln-Trp-Ala-Val-Gly-His-NH—CH[CH₂—CH(CH₃)₂]₂.

According to an embodiment, the radiolabeled GRPR-antagonist is radiolabeled NeoB2 of formula (III):

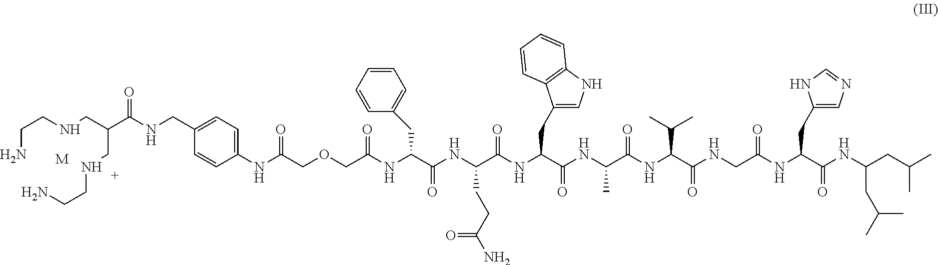

(M-N₄ (p-aminobenzylamine-diglycolic acid)-[D-Phe-Gln-Trp-Ala-Val-Gly-His-NH—CH[CH₂—CH(CH₃)₂]₂;

wherein M is a radonuclide.

According to another specific embodiment, the GRPR-antagonist is ProBOMB1 of the following formula (II):

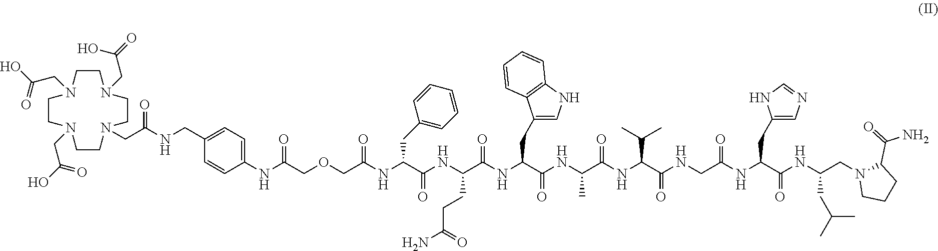

(DOTA-pABzA-DIG-D-Phe-Gln-Trp-Ala-Val-Gly-His-Leu-ψ(CH2N)-Pro-NH2)

Synthesis of the Compounds of Formula (I), (II) and (III)

The compounds of formula (I), (II), and (III) can be synthesized using the methods disclosed in the reference “Positron Emission Tomography Imaging of the Gastrin-Releasing Peptide Receptor with a Novel Bombesin Analogue” ACS Omega 2019, 4, 1470-1478.

The First Vial Comprising Said GRPR Antagonist

In certain embodiments, the radiolabeling method uses a single vial kit. In this embodiment, said first vial comprises said GRPR antagonist and a buffering agent, both in dried forms.

Alternatively, the radiolabeling method uses a two vial kit. In this embodiment, the first vial comprises said GRPR antagonist, and the second vial comprises the buffering agent.

For example, said GRPR antagonist, typically NeoB compound, is comprised in said first vial at an amount between 20 and 60 μg, typically, 50 μg.

Said first vial optionally comprises additional excipients such as radiolysis protector, bulking agent, and tensioactive agent.

In preferred embodiments, gentisic acid may be used as a radiolysis protector, preferably at an amount between 50 and 250 μg, typically, 200 μg.

In preferred embodiments, mannitol may be used as a bulking agent, for example at an amount between 10 and 30 mg, typically 20 mg.

In preferred embodiments, macrogol 15 hydroxystearate may be used as a surfactant, for example at an amount between 250 and 750 μg, typically 500 μg. Said surfactant advantageously reduced non-specific adhesion of the NeoB compound on glass or plastic surfaces, thereby optimizing the yield of the labeling process.

A preferred example of said first vial (Vial 1 of a two vial kit) is given in the examples.

The first vial is preferably obtained by freeze-drying using methods well known in the art. Therefore, said first vial may be provided in a lyophilized or spray dried form.

As used herein, the buffering agent is a buffer suitable for obtaining a pH from 3.0 to 6.0, preferably between 3.0 to 4.0 at the incubating step (iii). A “buffer for a pH from 3.0 to 6.0, preferably from 3.0 to 4.0” may advantageously be a formic acid buffer with hydroxide sodium.

Said buffering agent may further be comprised in the first vial, in an embodiment using a single vial kit, or a separate second vial, in an embodiment using a two vial kit.

Step (ii) of Adding a Solution of Said Radioactive Isotope into Said Fist Vial

Radioactive isotopes for use in the radiolabeling methods include those suitable as contrast agent in PET and SPECT imaging comprising the following:

¹¹¹In, ^{133m}In, ^{99m}Tc, ^{94m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁸⁶Y ⁵¹Cr, ^{52m}Mn ¹⁵⁷Gd, ¹⁶⁹Yb, ¹⁷²Tm, ^{117m}Sn, ⁸⁹Zr, ⁴³Sc, ⁴⁴Sc.

According to a preferred embodiment, the radioactive isotope is ⁶⁸Ga, ⁶⁷Ga or ⁶⁴Cu. In a preferred embodiment, ⁶⁷Ga is used for SPECT imaging and ⁶⁸Ga and ⁶⁴Cu are used for PET imaging such as PET/CT or PET/MRI.

The metallic ions of such radioisotopes are able to form non-covalent bond with the functional groups of the chelator, e.g. carborboxylic acids of the GRPR antagonist.

In a specific embodiment, said solution of said radioactive isotope is an eluate obtained from the steps of
i. producing a radioactive isotope from a parent non-radioactive element by means of a radioactive isotope generator,
ii. separating said radioactive isotope from said parent non-radioactive element by elution in HCl as an elution solvent,
iii. recovering the eluate,
   thereby obtaining a solution of said radioactive isotope in HCl.

Said solution comprising the radioactive isotope ⁶⁸Ga is an eluate typically obtained from the steps of:
i. producing producing ⁶⁸Ga element from a parent element ⁶⁸Ge, by means of a generator,
ii. optionally, separating the generated ⁶⁸Ga element from ⁶⁸Ge element by passing the elements ⁶⁸Ge/⁶⁸Ga through a suitable cartridge, and eluting ⁶⁸Ga in HCl,
   thereby obtaining a solution of said radioactive isotope in HCl.

Such methods of producing ⁶⁸Ga from ⁶⁸Ge/⁶⁸Ga generators are well-know in the art and for example described in Martiniova L,et al. Gallium-68 in Medical Imaging. Curr Radiopharm. 2016; 9(3):187-20; Dash A, Chakravarty Radionuclide generators: the prospect of availing PET radiotracers to meet current clinical needs and future research demands R Am J Nucl Med Mol Imaging. 2019 Feb. 15;9(1):30-66.

Said solution comprising the radioactive isotope ⁶⁸Ga may be an eluate preferably obtained from cyclotron production. Such production is for example described in Am J Nucl Med Mol Imaging 2014; 4(4):303-310 or in B. J. B. Nelson et al./Nuclear Medicine and Biology 80-81 (2020) 24-31.

Preferably, ⁶⁸Ga may be produced by a cyclotron, more preferably using a proton beam of energy between 8 and 18 MeV, even more preferably between 11 and 14 MeV. The ⁶⁸Ga may be produced via the ⁶⁸Zn(p,n)⁶⁸Ga reaction using a a solid or liquid target system. The target consists of enriched ⁶⁸Zn metal or ⁶⁸Zn liquid solution. After irradiation, the target is transferred for further chemical processing in which the ⁶⁸Ga is isolated using ion exchange chromatography. ⁶⁸Ga is eluted in HCl solution.

Alternatively, said radioactive isotope is ⁶⁷Ga. Various methods for the production of ⁶⁷Ga, using either a zinc (enriched or natural) or copper or germanium target with protons, deuterons, alpha particles or helium(III) as the bombarding particle, have been reported as summarised by Helus, F., Maier-Borst, W., 1973. A comparative investigation of methods used to produce 67Ga with a cyclotron. In: Radiopharmaceuticals and Labelled Compounds, Vol. 1, IAEA, Vienna, pp. 317-324, M. L Thakur Gallium-67 and indium-111 radiopharmaceuticals Int. J. Appl. Rad. Isot., 28 (1977), pp. 183-201, and Bjørnstad, T., Holtebekk, T., 1993. Production of ⁶⁷Ga at Oslo cyclotron. University of Oslo Report OUP8-3-1, pp. 3-5. Bombardment of ^{nat}Ge targets with moderate energy protons (up to 64 MeV) is also a suitable method to produce 67Ga as described in T Horiguchi, H Kumahora, H Inoue, Y Yoshizawa Excitation functions of Ge(p,xnyp) reactions and production of 68Ge, Int. J. Appl. Radiat. Isot., 34 (1983), pp. 1531-1535.

Preferably, ⁶⁷Ga may be produced by a cyclotron. Such methods of producing ⁶⁷Ga from ⁶⁸Zn (p, 2n)⁶⁷Ga are well-known in the art and for example described inAlirezapour B et al. Iranian Journal of Pharmaceutical Research (2013), 12 (2): 355-366. More preferably, this method uses a proton beam of energy between 10 and 40 MeV. The ⁶⁷Ga may be produced via either the ⁶⁷Zn (p, n)⁶⁷Ga or either the ⁶⁸Zn (p, 2n)⁶⁷Ga reaction using a solid or liquid target system. The target consisted of enriched ⁶⁷Zn or ⁶⁸Zn metal or liquid solution. After irradiation, the target is transferred for further chemical processing in which the ⁶⁷Ga is isolated using ion exchange chromatography. Final evaporation from aq. HCl yield ⁶⁷GaCl₃, which may then be added to said single vial for the labelling method.

Alternatively, said radioactive isotope is 64Cu as obtained from cyclotron production. Such production method is for example described in WO2013/029616.

Typically, ⁶⁴Cu may be produced by a cyclotron, preferably using a proton beam of energy between 11 and 18 MeV. The ⁶⁴Cu may be produced via the ⁶⁴Ni (p,n)⁶⁴Cu reaction using a solid or liquid target system. The target consisted of ⁶¹Ni metal or 64Ni liquid solution. After irradiation, the target is transferred for further chemical processing in which the ⁶⁴Cu is isolated using ion exchange chromatography. Final evaporation from aq. HCl yield ⁶⁴CuCl2, which may then be added to said first vial for the labelling method.

Step (iii) of Mixing the Solution Obtained in Step (ii) with at Least a Buffering Agent and Incubating it for a Sufficient Period of Time for Obtaining Said GRPR Antagonist Labeled with Said Radioactive Isotope

The radiolabelling starts after the mixing of first vial comprising the GRPR antagonist (e.g; the NeoB compound) with the solution comprising the radioactive isotope (typically, ⁶⁸Ga, ⁶⁷Ga or ⁶⁴Cu as disclosed above) in a suitable buffering agent as disclosed above.

In specific embodiments, the incubating step is performed at a temperature between 80° C. and 100° C., preferably between 90° C. and 100° C., typically at about 95° C.

In specific embodiments, the incubating step is performed for a period of time comprised between 5 and 10 minutes, for example between 6 and 8 minutes, typically about 7 minutes.

At the end of labeling process, a sequestering agent having a particular affinity for the radioactive isotope (such as 68Ga, ⁶⁷Ga or 64Cu) may be added to chelate the non-reacted part of the isotope. This complex formed by the sequestering agent and the non-reacted radioactive isotope may then be discarded to increase the radiochemical purity after radiolabelling.

Preferred Embodiments of the Methods for Radiolabelling NeoB with ⁶⁸Ga

The present disclosure more particularly relates to a method for labeling NeoB compound of formula (I) with ⁶⁸Ga,

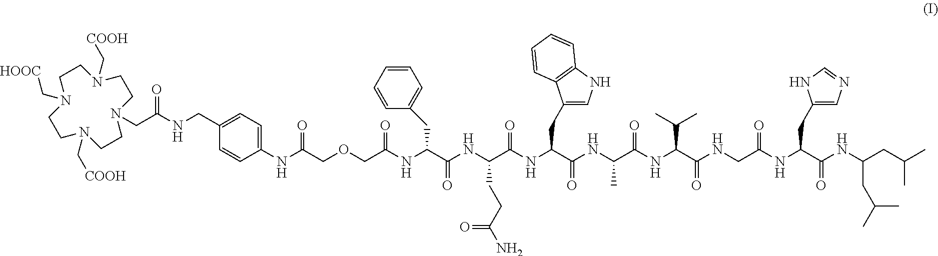 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

(DOTA-(p-aminobenzylamine-diglycolic acid))-[D-Phe-Gln-Trp-Ala-Val-Gly-His-NH—CH[CH₂—CH(CH₃)₂]₂; said method comprising the steps of:
i. providing a first vial containing about 50 μg of NeoB and between 50 and 250 μg of gentisic acid, in dried forms,
   ii. adding a solution of ⁶⁸Ga in HCl to said first vial,
   iii. mixing the solution obtained in ii. with a buffering agent for adjusting the pH in a range of 3.0 and 4.0, and incubating it for a sufficient period of time for obtaining said NeoB compound labeled with ⁶⁸Ga,
   iv. optionally adjusting the pH of the solution.

In specific embodiments of said methods, said solution of said ⁶⁸Ga in HCl is an eluate obtained from the steps of
i. producing ⁶⁸Ga element from a parent element ⁶⁸Ge, by means of a generator,
ii. optionally, separating the generated ⁶⁸Ga element from ⁶⁸Ge element by passing the elements ⁶⁸Ga/⁶⁸Ge through a suitable cartridge, and eluting ⁶⁸Ga in HCl,
   thereby obtaining a solution of said radioactive isotope in HCl.

Typically, said buffering agents consist of 60 mg of formic acid and 56.5 mg of sodium hydroxide.

Advantageously, in specific embodiments, a simple labelling of the GRPR antagonist may be obtained with an eluate of ⁶⁸Ga in HCl coming from commercially available ⁶⁸Ge/⁶⁸Ga generators without any processing of the eluate or any additional purification step.

Powder for Solution for Injection

The disclosure further relates to a powder for solution for injection, comprising the following components in dried forms:
i. A GRPR antagonist as defined above, typically NeoB of formula (I) as defined above;
ii. a radiolysis protector, for example gentisic acid;
iii. a bulking agent, for example, mannitol; and,
iv. optionally a surfactant, for example macrogol 15 hydroxystearate.

A preferred embodiment comprises the following components:
i. NeoB of the following formula (I) at an amount between 20 and 60 μg, typically, 50 μg;

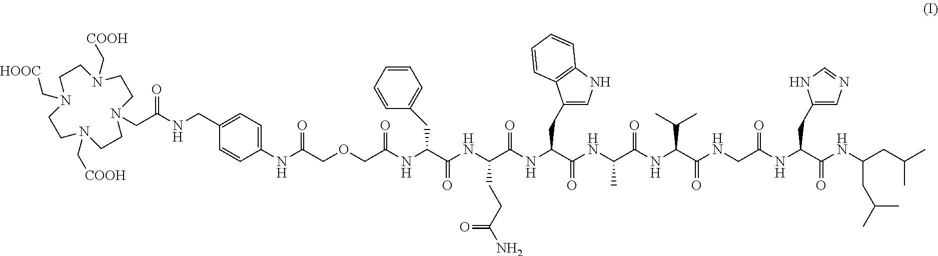 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

ii. gentisic acid at an amount between 50 and 250 μg, typically, 200 μg, and,
iii. mannitol at an amount between 10 and 30 mg, for example 20 mg, and,
iv. macrogol 15 hydroxystearate at an amount between 250 and 750 μg, for example 500 μg.

Radiolabelling Kits of the Disclosure

The present disclosure also relates to a kit for carrying out the above labeling methods, said kit comprising
i. a first vial with the following components in dried forms
   i. a GRPR antagonist as defined above,
   ii. a radiolysis protector, for example gentisic acid,
   iii. optionally a bulking agent, for example, mannitol, and,
   iv. optionally a surfactant, for example macrogol 15 hydroxystearate; and,
ii. a second vial comprising at least a buffering agent, preferably in dried form; and,
iii. optionally, an accessory cartridge for eluting a radioactive isotope generated by a radioactive isotope generator.

Preferably, said first or single vial comprises the following components:
i. NeoB of the following formula (I) at an amount between 20 and 60 μg, typically, 50 μg:

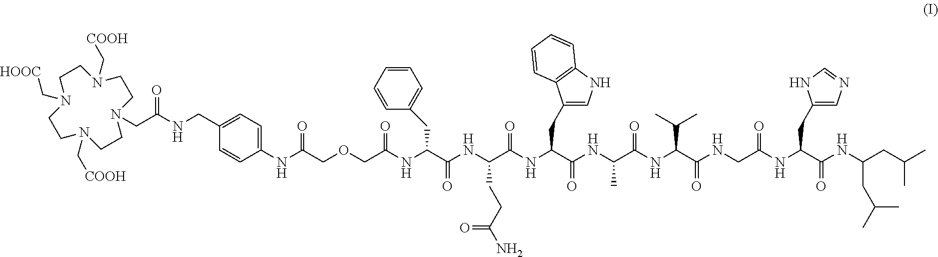 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

ii. gentisic acid at an amount between 50 and 250 μg, typically, 200 μg,
iii. mannitol at an amount between 10 and 30 mg, for example 20 mg, and,
iv. optionally, macrogol 15 hydroxystearate at an amount between 250 and 750 μg, for example 500 μg.

Said second vial or single vial may comprise buffering agents for maintaining a pH between 3.0 and 4.0. For example, said second vial comprises formic acid and sodium hydroxide as buffering agents.

Preferably, all components of said first, second or single vial are in dried forms.

The radioactive isotope for labeling the GRPR antagonist may be provided with the kit as ready-for-use product, i.e. for mixing and incubating with the first vial and buffering agent as provided by the kit, or alternatively may be eluted from a radioactive isotope generator prior to, and shortly before mixing and incubating with said first vial and buffering agent, particularly in cases said radioactive isotope has a relatively short half-life such as ⁶⁸Ga, ⁶⁷Ga and ⁶⁴Cu.

Preferably, the components are inserted into sealed containers which may be packaged together, with instructions for performing the method according to the present disclosure.

The kit can also be used as a part of an automatic system or a remotely controlled mechanism system that automatically performs the elution of the gallium-69 generator and/or the subsequent mixing and heating. In this embodiment, the vial containing the GRPR antagonist (first vial) is directly connected to the elution system and/or the heating system

The kit may be applied in particular for use in the methods as disclosed in the next section.

In specific embodiments, the GRPR-antagonist is NeoB as defined above.

Use of the Kit According to the Present Disclosure

The above-defined kits may be applied in particular for use of the labeling methods as disclosed in the previous sections.

Advantageously, a solution comprising a GRPR antagonist (e.g. NeoB compound) labeled with a radioactive isotope (for example ⁶⁸Ga, ⁶⁷Ga or ⁶⁴Cu) is obtainable or obtained by the labeling methods as disclosed in the previous sections.

Such solution may be ready for use as an injectable solution, for example, for in vivo detection of tumors by imaging in a subject in need thereof.

In certain aspects the subject is a mammal, for example but not limited to a rodent, canine, feline, or primate. In preferred aspects, the subject is a human.

The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and \hat{ }SHP Handbook on Injectable Drugs, Trissel, 15th ed., pages 622-630 (2009)).

Typically, said solution for use as an injectable solution provides a single dose between 150-250 MBq of [68Ga]-NeoB for administration to a subject in need thereof.

In specific embodiments, said subject in need thereof a subject has cancer, more specifically, is a patient that has tumor selected from prostate cancer, breast cancer, small cell lung cancer, colon carcinoma, gastrointestinal stromal tumors, gastrinoma, glioma, glioblastoma, renal cell carcinomas, gastroenteropancreatic neuroendocrine tumors, oesophageal squamous cell tumors, neuroblastomas, head and neck squamous cell carcinomas, as well as ovarian, endometrial and pancreatic tumors displaying neoplasia-related vasculature that may be GRPR-positive.

Typically, PET/MRI, SPECT or PET/CT imaging may be performed between 1 h and 4 hours after the administration of the radiolabelled GRPR antagonist to the subject, and more preferably with 2 and 3 hours after the administration of the radiolabelled GRPR antagonist to the subject.

### Embodiments

The following specific embodiments are disclosed
1. A method for labeling a gastrin-releasing peptide receptor (GRPR) antagonist with a radioactive isotope, preferably ⁶⁸Ga, ⁶⁷Ga or ⁶⁴Cu, said method comprising the steps of:
   i. providing a first vial comprising said GRPR antagonist in dried form,
   ii. adding a solution of said radioactive isotope into said first vial, thereby obtaining a solution of said GRPR antagonist with said radioactive isotope,
   iii. mixing the solution obtained in ii. with at least a buffering agent and incubating it for a sufficient period of time for obtaining said GRPR antagonist labeled with said radioactive isotope, and,
   iv. optionally, adjusting the pH of the solution.
2. The method of Embodiment 1, wherein the first vial at step i. is a reaction vial comprising said GRPR antagonist and a buffering agent, preferably both in dried forms.
3. The method of Embodiment 1, wherein step iii comprises mixing the solution obtained in ii. with at least a reaction solution comprising a buffering agent and incubating it for a sufficient period of time for obtaining said GRPR antagonist labeled with said radioactive isotope.
4. The method of any one of Embodiments 1-3, wherein said solution with said radioactive isotope further comprises HCl.
5. The method of any one of Embodiments 1-4, wherein said radioactive isotope is ⁶⁸Ga and the radiochemical purity as measured in HPLC is at least 92%, and optionally, the percentage of free ⁶⁸Ga3+ (in HPLC) is 2% or less, and/or the percentage of non-complexed ⁶⁸Ga3+ species (in ITLC) is 3% or less.
6. The method of any one of Embodiments 1-4, wherein said radioactive isotope is ⁶⁷Ga and the radiochemical purity as measured in HPLC is at least 92%, and optionally, the percentage of free ⁶⁷Ga 3+ (in HPLC) is 2% or less, and/or the percentage of non-complexed ⁶⁷Ga 3+ species (in ITLC) is 3% or less.
7. The method of any one of Embodiments 1-4, wherein said radioactive isotope is ⁶⁴Cu and the radiochemical purity as measured in HPLC is at least 92%, and optionally, the percentage of free ⁶⁴Cu2+ (in HPLC) is 2% or less, and/or the percentage of non-complexed ⁶⁴Cu2+ species (in ITLC) is 3% or less.
8. The method of any one of Embodiments 1-7, wherein said GRPR antagonist is a compound of the following formula:

C—S—P
wherein:
C is a chelator capable of chelating said radioactive isotope;
S is an optional spacer covalently linked between C and the N-terminal of P;
P is a GRPR peptide antagonist, preferably of the general formula:

Xaa1-Xaa2-Xaa3-Xaa4Xaa5-Xaa6-Xaa7-Z;
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) (all as L- or D-isomers);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl)histidine (3-Me)His;
Z is selected from —NHOH, —NHNH2, —NH-alkyl, —N(alkyl)2, and —O-alkyl or Z is

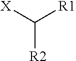

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido, or amide substituted aryl or heteroaryl group; and,
9. The method of Embodiment 8, wherein P is DPhe-Gln-Trp-Ala-Val-Gly-His-NH—CH(CH₂—CH(CH₃)₂)₂.
10. The method of Embodiment 8, wherein the GRPR antagonist is NeoB compound of formula (I):

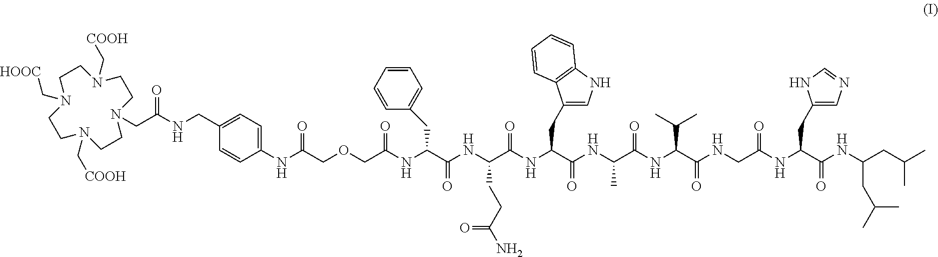 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

DOTA-(p-aminobenzylamine-diglycolic acid))-D-Phe-Gln-Trp-Ala-Val-Gly-His-NH—CH[CH₂—CH(CH₃)₂]₂
11. The method of any one of Embodiments 1-10, wherein said GRPR antagonist is comprised in said first vial at an amount between 20 and 60 μg, typically, 50 μg.
12. The method of any one of Embodiments 1-11, wherein said first vial further comprises gentisic acid as a radiolysis protector, preferably at an amount between 50 and 250 μg, typically, 200 μg.
13. The method of any one of Embodiments 1-12, wherein said first vial further comprises mannitol as a bulking agent, for example at an amount between 10 and 30 mg, typically 20 mg.
14. The method of any one of Embodiments 1-13, wherein said first vial further comprises a macrogol 15 hydroxystearate as a surfactant, for example at an amount between 250 and 750 μg, typically 500 μg.
15. The method of any one of Embodiments 1-14, wherein said buffering agent is present in an amount suitable for obtaining a pH between 3.0 at the incubating step (iii).
16. The method of any one of Embodiments 1-15, wherein said buffering agent comprises formic acid and hydroxide sodium.
17. The method of any one of Embodiments 1-16, wherein the incubating step is performed at a temperature between 80° C. and 100° C., preferably between 90° C. and 100° C., typically at about 95° C.
18. The method of any one of Embodiments 1-17, wherein the incubating step is performed for a period of time comprised between 5 and 10 minutes, for example between 6 and 8 minutes, typically about 7 minutes.
19. The method of any one of Embodiments 1-18, wherein said solution of said radioactive isotope is an eluate obtained from the steps of
   i. producing a radioactive isotope from a parent non-radioactive element by means of a radioactive isotope generator,
   ii. separating said radioactive isotope from said parent non-radioactive element by elution in HCl as an elution solvent,
   iii. recovering the eluate,
      thereby obtaining a solution of said radioactive isotope in HCl.
20. A method for labeling NeoB compound of formula (I) with ⁶⁸Ga,

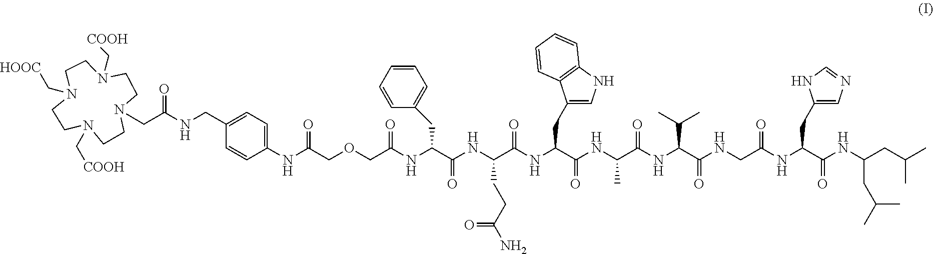 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

(DOTA-(p-aminobenzylamine-diglycolic acid))-D-Phe-Gln-Trp-Ala-Val-Gly-His-NH—CH[CH₂—CH(CH₃)₂]₂ said method comprising the steps of:
i. providing a first vial containing about 50 g of NeoB and between 50 and 250 μg of gentisic acid, in dried forms,
ii. adding a solution of ⁶⁸Ga in HCl to said first vial,
iii. mixing the solution obtained in ii. with a buffering agent for adjusting the pH in a range of 3.0 and 4.0, and incubating it for a sufficient period of time for obtaining said NeoB compound labeled with ⁶⁸Ga,
iv. optionally adjusting the pH of the solution.
21. The method of Embodiment 20, wherein said solution of said ⁶⁸Ga in HCl is an eluate obtained from the steps of
i. producing ⁶⁸Ga element from a parent element ⁶⁸Ge, by means of a generator,
ii. optionally, separating the generated ⁶⁸Ga element from ⁶⁸Ge element by passing the elements ⁶⁸Ga/⁶⁸Ge through a suitable cartridge, and eluting ⁶⁸Ga in HCl,
   thereby obtaining a solution of said radioactive isotope in HCl.
22. The method of Embodiment 20 or 21, wherein said buffering agents consist of 60 mg of formic acid and 56.5 mg of sodium hydroxide.
23. The method of any one of Embodiments 20-22, wherein the incubating step is performed at a temperature between 80° C. and 100° C., preferably between 90° C. and 100° C., typically at about 95° C.
24. The method of any one of Embodiments 20-23, wherein the incubating step is performed for a period of time comprised between 5 and 10 minutes, for example between 6 and 8 minutes, typically about 7 minutes.
25. A solution comprising a GRPR antagonist labeled with a radioactive isotope, obtainable or obtained by the method of any one of Embodiments 1-24, for use as an injectable solution for in vivo detection of tumors by imaging in a subject in need thereof.
26. A solution comprising NeoB compound labeled with ⁶⁸Ga, obtainable or obtained by the method of any one of Embodiments 20-24, for use as an injectable solution for in vivo detection of tumors by imaging in a subject in need thereof.
27. The solution for use according to Embodiment 25 or Embodiment 26, wherein said tumors are selected from GRPR expressing tumors, preferably said GRPR expressing tumors is selected from prostate cancer, breast cancer, small cell lung cancer, colon carcinoma, gastrointestinal stromal tumors, gastrinoma, renal cell carcinomas, gastroenteropancreatic neuroendocrine tumors, oesophageal squamous cell tumors, neuroblastomas, head and neck squamous cell carcinomas, as well as ovarian, endometrial and pancreatic tumors displaying neoplasia-related vasculature that is GRPR positive.
28. A powder for solution for injection, comprising the following components in dried forms:
   i. A GRPR antagonist of the following formula:

C—S—P
wherein:
C is a chelator capable of chelating said radioactive isotope;
S is an optional spacer covalently linked between C and the N-terminal of P;
P is a GRPR peptide antagonist preferably of the general formula:

Xaa1-Xaa2-Xaa3-Xaa4Xaa5-Xaa6-Xaa7-Z;
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (o-I-Tyr), Trp and pentafluorophenylalanine (5-F-Phe) (all as L- or D-isomers);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or j-Ala;
Xaa7 is His or (3-methyl)histidine (3-Me)His;
Z is selected from —NHOH, —NHNH2, —NH-alkyl, —N(alkyl)2, and —O-alkyl or Z is

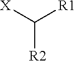

wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido, or amide substituted aryl or heteroaryl group;
ii. a radiolysis protector, for example gentisic acid;
iii. a bulking agent, for example, mannitol; and,
iv. optionally a surfactant, for example macrogol 15 hydroxystearate.
29. The powder for solution for injection according to Embodiment 28, wherein said GRPR antagonist is NeoB compound of formula (I) below:

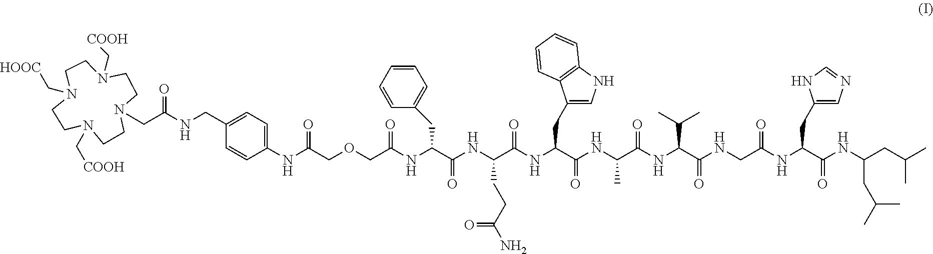 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

30. The powder for solution for injection according to Embodiment 29, wherein the NeoB compound is comprised at an amount between 20 and 60 μg, typically, 50 μg.
31. The powder for solution for injection of any one of Embodiments 28-30, wherein the gentisic acid is comprised at an amount between 50 and 250 μg, typically, 200 μg.
32. The powder for solution for injection of any one of Embodiments 28-31, wherein said bulking agent is mannitol at an amount between 10 and 30 mg, for example 20 mg.
33. The powder for solution for injection of any one of Embodiments 28-32, wherein said surfactant is macrogol 15 hydroxystearate at an amount between 250 and 750 μg, for example 500 μg.
34. The powder for solution for injection of any one of Embodiments 28-33, comprising the following components:
   NeoB of the following formula (I) at an amount between 20 and 60 μg, typically, 50 μg;

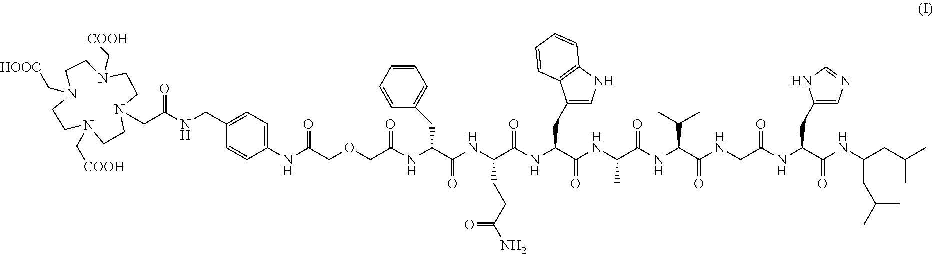 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

gentisic acid at an amount between 50 and 250 μg, typically, 200 μg, and,
mannitol at an amount between 10 and 30 mg, for example 20 mg, and,
macrogol 15 hydroxystearate at an amount between 250 and 750 μg, for example 500 μg.
35. A kit for carrying out the method of Embodiment 20, comprising
   i. a first vial with the following components in dried forms
      NeoB of the following formula (I):

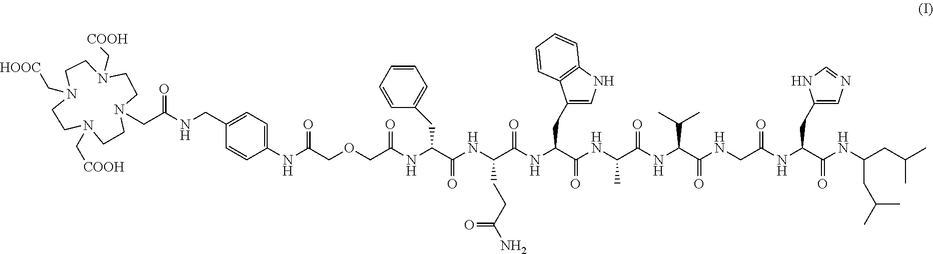 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

a radiolysis protector, for example gentisic acid,
optionally a bulking agent, for example, mannitol, and,
optionally a surfactant, for example macrogol 15 hydroxystearate; and,
ii. a second vial comprising at least a buffering agent, preferably in dried form; and,
iii. optionally, an accessory cartridge for eluting a radioactive isotope generated by a radioactive isotope generator.
36. A kit for carrying out the method of Embodiment 20, comprising
   i. a single vial with the following components in dried forms
      NeoB of the following formula (I):

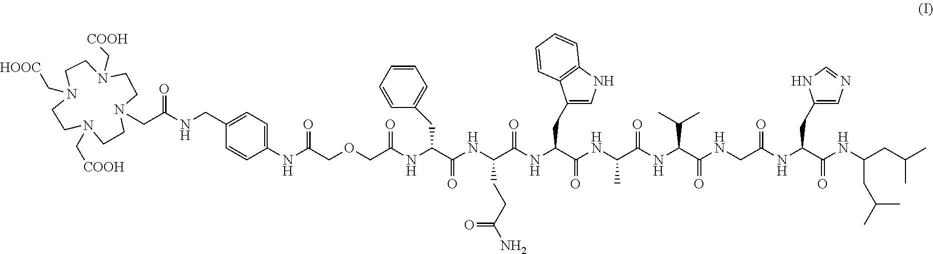 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

a radiolysis protector, for example gentisic acid,
optionally a bulking agent, for example, mannitol,
optionally a surfactant, for example macrogol 15 hydroxystearate, and,
at least a buffering agent, preferably in dried form; and,
ii. optionally, an accessory cartridge for eluting a radioactive isotope generated by a radioactive isotope generator.
37. The kit of Embodiment 35 or 36, wherein the NeoB compound is comprised at an amount between 20 and 60 μg, typically, 50 μg.
38. The kit of any one of Embodiments 35-37, wherein the gentisic acid is comprised at an amount between 50 and 250 μg, typically, 200 μg.
39. The kit of any one of Embodiments 35-38, wherein said bulking agent is mannitol at an amount between 10 and 30 mg, for example 20 mg.
40. The kit of any one of Embodiments 35-39, wherein said surfactant is macrogol 15 hydroxystearate at an amount between 250 and 750 μg, for example 500 μg.
41. The kit of any one of Embodiments 35-40, wherein said first or single vial comprises the following components:
   NeoB of the following formula (I) at an amount between 20 and 60 μg, typically, 50 μag;

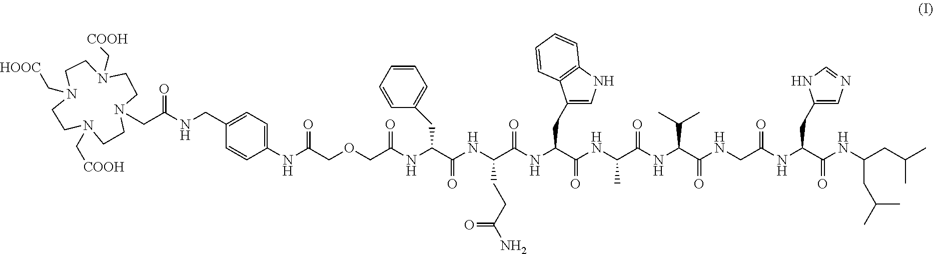 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

gentisic acid at an amount between 50 and 250 μg, typically, 200 μg,
mannitol at an amount between 10 and 30 mg, for example 20 mg, and,
optionally, macrogol 15 hydroxystearate at an amount between 250 and 750 μg, for example 500 μg.
42. The kit of any one of Embodiments 35-41, wherein said second vial or single vial comprises buffering agents for maintaining a pH between 3.0 and 4.0.
43. The kit any one of Embodiments 35-42, wherein said second vial comprises formic acid and sodium hydroxide as buffering agents.
44. The kit of any one of Embodiments 35-43, wherein all components of said first, second or single vial are in dried forms.

### EXAMPLES

Hereinafter, the present disclosure is described in more details and specifically with reference to the examples, which however are not intended to limit the present invention.

Radiochemical Purity by ITLC

Preparation of the Mobile Phase Solutions:

Ammonium acetate 5M: Accurately weigh 3.85 g (3.84615÷3.85385 g) of ammonium acetate in a graduate flask of 10 mL and dissolve it with 10 mL of MilliQ water.

Ammonium acetate/MeOH: Using a graduated cylinder, add 1 mL of the ammonium acetate solution 5 M, 2 mL of MilliQ water and 7 mL of methanol. Transfer the eluent in the TLC chamber.

ITLC-SA preparation: Cut one ITLC-SA per each vial of 115 mm, draw a line at 20 mm from the bottom (where put a 5 uL drop of sample) and draw a line at 100 mm from the bottom (where the chromatographic development must give up).

⁶⁸Ga-NeoB: reference factor 0.6-0.9

⁶⁸Ga non-complexed species: reference factor=0.0÷0.1

(⁶⁸Ga non-complexed species refers to ⁶⁸Ga colloidal species and ⁶⁸Ga free.)

Radiochemical Purity by HPLC

TABLE 1
Chromatographic conditions

| Flow rate | 1.0 mL/min | | | |
|---|---|---|---|---|
| Column | Acris PEPTIDE 3.6u XB-C18- | | | |
| type | New Column 150 × 4.6 mm | | | |
| | Security Guard Ultra Cartridge | | | |
| | UHPLC C18 Peptide (Guard Column) | | | |
| Injection | 20 μL | | | |
| volume | | | | |
| Detector | Radiometric Gabi Star-UV (278 nm) | | | |
| Pump program | Time (min) | A (%) | B (%) | Curve |
| | 0-2 | 85 | 15 | 0 |
| | 2-9 | 60 | 40 | 1 |
| | 9-11 | 60 | 40 | 0 |
| | 11-11.5 | 0 | 100 | 1 |
| | 11.5-13 | 0 | 100 | 0 |
| | 13-13.1 | 85 | 15 | 1 |
| | 13.1-15.5 | 85 | 15 | 0 |

| | |
|---|---|
| Retention | free ⁶⁸Ga: ~1.5 |
| time (min) | ⁶⁸Ga-NeoB: ~10.4 |
| Detection | 13 min |
| time | |
| Run time | 15.5 min |

### Example 1: Development of a Method for Radiolabeling NeoB with 68Gallium Using a Two-Vial Kit

1. Description and Composition of the 2-Vial Kit

The applicant developed a sterile 2-vial kit which consists of:
Vial 1: NeoB, 50 μg, powder for solution for injection, to be reconstituted with a solution of gallium-68 chloride (⁶⁸GaCl₃) in HCl eluted from a ⁶⁸Ge/⁶⁸Ga generator;
Vial 2: Reaction buffer.

Vial 2 is to be added to the reconstituted Vial 1.

One accessory cartridge is used to reduce the amount of germanium-68 (68Ge) ions potentially present in the generator eluate.

The kit has to be used in combination with a solution of ⁶⁸Ga in HCl provided by a ⁶⁸Ge/⁶⁸Ga generator to obtain ⁶⁸Ga-NeoB solution for injection, being the Radiolabelled Imaging Product, which can be directly injected to the patient.

The volume of ⁶⁸Ga-NeoB solution for injection, corresponding to the radioactive dose to be administered, is calculated according to the estimated time of injection, on the basis of the current activity provided by the generator and of physical decay of the radionuclide (half-life=68 min). It is a mono-dose product.

Vial 1 is a powder for solution for injection containing 50 μg NeoB as active ingredient, packed in 10 mL glass vial.

The composition of Vial 1 is provided in Table 2.

TABLE 2
Composition of Vial 1 powder for solution for injection

| | Composition | | |
|---|---|---|---|
| Component | (per vial) | | Function |
| NeoB | 50 | μg | Drug Substance |
| Gentisic acid | 200 | μg | Radiolysis |
| | | | protector/Antioxidant |
| Mannitol | 20 | mg | Bulking agent |
| Macrogol 15 | 500 | μg | Tensioactive agent |

hydroxystearate
(Kolliphor HS 15)

The composition of Vial 2 is provided in Table 3.

TABLE 3

| | Composition | |
|---|---|---|
| Component | (per vial) | Function |
| Formic acid | 60 mg | Buffering agent |
| Sodium hydroxide | 56.5 mg | Buffering agent |
| Water for injection | Qs to 1 mL | Solvent |

2. Pharmaceutical Development

As described above, Vial 1 (NeoB, 50 μg, powder for solution for injection) is part of a radiopharmaceutical kit which also contain a reaction buffer (Vial 2) and an accessory cartridge.

The kit has to be used in combination with a solution of ⁶⁸Ga in HCl provided by a ⁶⁸Ge/⁶⁸Ga generator to obtain ⁶⁸Ga-NeoB solution for injection, being the Radiolabelled Imaging Product, which can be directly injected to the patient.

2.1 Components of the Drug Product

The drug product contains NeoB as active ingredient and gentisic acid, mannitol and Kolliphor HS 15 as excipients.

2.1.1 Drug Substance

The active substance is the NeoB peptide, a 7-meraminoacid sequence covalently bound to a chelator (DOTA) through the PABZA-DIG linker, as shown in Formula (I) below:

DOTA PABZA_{D}Phe⁵GIn⁷Trp⁸Ala⁹Val¹⁰Gly¹¹HIsNHCH[(CH₂—CH(CH₃)₂]¹²

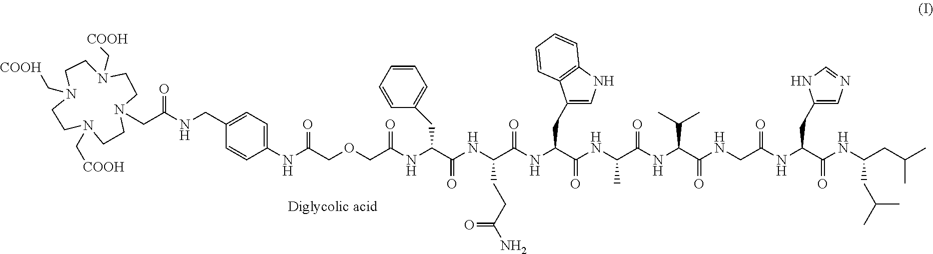

2.1.2 Excipients

The excipients chosen for the composition of Vial 1 are added to maintain stability of the active substance in the final formulation, to assure safety and efficacy of the drug product and also to obtain the required radiochemical purity of the ⁶⁸Ga-NeoB solution during the reconstitution procedure. The excipients selected lead to a drug product with the required pharmaco-technical characteristics.

The non-pharmacopoeial excipient gentisic acid with specific function was added in the drug product composition, linked to the purity and the stability of the Radiolabelled Imaging Product obtained after reconstitution.

A brief description of each excipient is provided as follows:
Mannitol
   Mannitol is used as bulking agent. Since peptide drugs are very potent, very small quantities are required in the drug product. In the absence of a bulking agent, the product processing becomes not suitable from technological point of view. Bulking agents allow pharmaceutical processing and the production of a presentable lyophilisate product.
Gentisic acid

Gentisic acid is a non-compendial excipient used as antioxidant in the drug product formulation.
Kolliphor HS 15 (Macrogol 15 hydroxystearate)

Kolliphor HS 15 is a water-soluble non-ionic solubilizer used in parenteral formulations. As a solubilizer, it is particularly suitable for parenteral and oral dosage forms.

Due to the non-specific binding of the peptide used as active ingredient in the NeoB radiopharmaceutical kit, Kolliphor HS 15 is used as tensioactive agent for the peptide who has the tendency to stick on the glass and plastic surfaces. As a non-ionic surfactant there is no risk of interfering during the labeling with ⁶⁸Ga.

2.2 Drug Product

2.2.1 Formulation Development

The formulation development has been performed with the aim of identifying the reaction mixture composition able to allow a simple labelling of the DOTA-peptide based on direct reconstitution with the eluate coming from commercially available ⁶⁸Ge/⁶⁸Ga generators without any processing of the eluate or any additional purification step.

The goal was to develop a Bombesin-like peptide antagonist (NeoB) to be used as radiotracer for the detection of GRPR-positive tumors.

Vial 1 is a lyophilisate powder containing the peptide as active ingredient which is radiolabeled with ⁶⁸Ga during the radiolabelling procedure.

Initial efforts to develop a suitable formulation for NeoB (Vial 1) have involved tests on the bulk solution prior to the sterilization and lyophilisation process prepared at a lab scale.

The development work was focused on the selection of the appropriate excipients in relation with the peptide characteristics in order to obtain a finished product that will conduct to a ⁶⁸Ga-radiolabelled NeoB product having the targeted radiochemical purity as follows:
⁶⁸Ga-NeoB (HPLC)→>92%
free ⁶⁸Ga³⁺ (HPLC)→≤2%
Non-complexed ⁶⁸Ga³⁺ species (ITLC)→≤3%

The components selected for the final formulation are as follows:

TABLE 4
Selected components for the Vial 1 composition (powder
for solution for injection)

| Component of Vial l composition | Function |
|---|---|
| NeoB peptide | Drug Substance |
| Gentisic acid | Radiolysis protector/ |
| | Antioxidant |
| Mannitol | Bulking agent |
| Macrogol 15 hydroxy stearate | Tensioactive agent |
| (Kolliphor HS 15) | |

The development work including the relevant performed studies is described starting from the selection of the active ingredient amount and appropriate excipients.

2.2.1.1 Selection of the Peptide Amount

Using the eluate coming from a 1850 MBq ⁶⁸Ge/⁶⁸Ga generator and the formate buffer, increasing amounts of NeoB peptide (from 15 μg up to 100 μg) were tested in the labeling procedure with the aim to identify the minimum amount of peptide necessary to have a ⁶⁸Ga incorporation above 98% in HPLC and 97% in ITLC. Based on the results summarized in Table 5, 25 μg is the lowest amount of peptide that gives reproducibility by good radiochemical purity.

TABLE 5
NeoB amount-effect on labelling efficiency

| | | | ITLC | |
|---|---|---|---|---|
| DOTA- | HPLC | | | % non- |
| peptide | % ⁶⁸Ga- | % free | % ⁶⁸Ga- | complexed |
| (μg) | NeoB | ⁶⁸Ga³⁺ | NeoB | ⁶⁸Ga³⁺ |
| 15 | 94.5% | 1.8% | 99.2% | 0.8% |
| 25 | 94.1% | 1.4% | 99.3% | 0.7% |
| 50 | 94.8% | 1.2% | 99.7% | 0.3% |
| 75 | 96.3% | 1.1% | 99.4% | 0.6% |
| 100 | 96.3% | 0.9% | 100.0% | 0.0% |

In parallel, different peptide doses were also tested in in vivo biodistribution experiment. Briefly, using a prostate cancer model in mice, two different peptide mass doses were compared: 10 pmol versus 200 pmol; the amount of total injected radioactivity was maintained constant in these experiments (1 MBq). Injection of the radiolabeled NeoB resulted in an increased accumulation in the tumour when the higher peptide mass dose was used (200 pmol). At the same time, the uptake in non-target organs (such as the pancreas) was considerably lower with the higher peptide mass does (200 pmol). Therefore, from these pre-clinical evaluations, it was demonstrated that higher peptide mass doses are to be preferred, since they are associated with decrease of uptake in non-target organs (especially the pancreas in this case).

Based on the radiolabelling tests performed (as described in Table 5) and on the in vivo biodistribution experiment that indicates that a higher peptide mass dose guarantees better performances and safety profile of the compound, the final amount of peptide selected to be included in Vial 1 was 50 μg.

The formulation development work also focused on the choice of the tensioactive, the antioxidant agent and the bulking agent. The radiolabeling procedure has also been thoroughly evaluated.

2.2.1.2 Selection of the Critical Excipients
Selection of the Tensioactive Agent

During the tests performed to define the formulation of the Vial 1 (NeoB 50 μg, powder for solution for injection), it appeared that the peptide has a particular tendency to stick on glass and plastic surfaces. This phenomenon is called non-specific binding (NSB). Peptides often demonstrate greater NSB issues than small molecules, especially uncharged peptides can strongly stick to plastics. The causes may be different: Physical/chemical properties, Van der Waals interactions, ionic interactions. Therefore, the addition of excipients known to reduce the NSB, including tensioactive and solubilizing agents, was assessed.

Organic solvent may enhance solubility and prevent adsorption. Ethanol for example, can be used in radiopharmaceutical injections to enhance the solubility of highly lipophilic tracers or to decrease adsorption to vials, membrane filters, and injection syringes. Ethanol could not be a choice in the case of NeoB powder for solution for injection, because it is not compatible with the freeze-drying process.

Human Serum Albumin (HSA) is also used in a number of protein formulations as a stabilizer to prevent surface adsorption but this excipient in not suitable due to its thermal instability.

Another possible approach in order to diminish the peptide non-specific binding was the use of surfactants (e.g Polysorbate 20, Polysorbate 80, Pluronic F-68, Sorbitan trioleate). Specific attention was given on the study of non-ionic surfactants, because the ionic surfactants may interfere with the labeling of ⁶⁸Ga.

Non-ionic tensioactives, like Kolliphor HS 15, Kolliphor K188, Tween 20, Tween 80, Polyvinylpyrrolidone K10, are commercially available as solubilizing excipients in oral and injectable formulations.

Peptide adhesion tests were performed with different tensioactive agents in order to evaluate the suitability of the most appropriate agent that can be used in the composition of NeoB powder for solution for injection (Vial 1) (see results in Table 6 below).

Hydroxy Propyl β Cyclodextrin was also assessed in the formulation, either alone or in combination with the tensioactive agent. As reported in below, the presence of Hydroxy Propyl β Cyclodextrin had only a limited positive impact on peptide adhesion. Furthermore, as demonstrated in subsequent tests (see also Section 2.2.1.3 Radiolabelling Procedure), the presence of Hydroxy Propyl β Cyclodextrin together with the tensioactive agent does not improve the radiochemical purity of the final product, if compared with a formulation containing the tensioactive agent only. For this reason, Hydroxy Propyl β Cyclodextrin was not included in the final formulation.

TABLE 6
Selection of tensioactive agents-peptide adhesion tests

| | | Adhesion |
|---|---|---|
| NeoB (μg) | Solubilizing agent (μg) | (%) |
| 50 | No tensioactive | 21.4 |
| 50 | PEG 300 (500 μg) | 15.0 |
| 50 | EtOH (80%) | 7.8 |
| 50 | DMSO (15%) | 10.4 |
| 50 | ACN (80%) | 7.9 |
| 50 | PEG 400 (500 μg) | 15.1 |
| 50 | PVP K10 (500 μg) | 15.6 |
| 50 | Albumin (500 μg) | 7.9 |
| 50 | Kolliphor P188 (1500 μg) | 7.9 |
| 50 | Hydroxy Propyl β Cyclodextrin (5000 μg) | 13.5 |
| 50 | PEG 4000 (500 μg) | 12.8 |
| 50 | Tween 20 (500 μg) | 6.1 |
| 50 | Kolliphor HS 15 (500 μg) | 6.2 |

The best results in term of peptide adhesion were obtained with the Kolliphor HS 15 and with Tween 20. The two excipients were further investigated to determine the final amount into the kit. The results obtained were good in term of radiochemical purity and peptide adhesion.

TABLE 7
Comparison between Tween 20 and Kolliphor HS 15
tensioactive agents

| | | HPLC ⁶⁸Ga- | Adhesion |
|---|---|---|---|
| NeoB (μg) | Tensioactive (mg) | NeoB (%) | (% ) |
| 50 | Tween 20 (1.5 mg) | 94.3 | 5.2 |
| 50 | Tween 20 (0.5 mg) | 94.7 | 6.0 |
| 50 | Tween 20 (0.1 mg) | 95.3 | 7.6 |
| 50 | Kolliphor HS 15 (2 mg) | 92.8 | 5.9 |
| 50 | Kolliphor HS 15 (1.5 mg) | 93.4 | 4.9 |
| 50 | Kolliphor HS 15 (1.0 mg) | 94.3 | 4.6 |
| 50 | Kolliphor HS 15 (0.5 mg) | 94.9 | 4.2 |
| 50 | Kolliphor HS 15 (0.25 mg) | 95.1 | 6.0 |
| 50 | Kolliphor HS 15 (0.1 mg) | 96.1 | 8.3 |
| 50 | Kolliphor HS 15 (0.05 mg) | 95.7 | 9.0 |

The final choice was Kolliphor HS 15 because the polysorbates (tween 20) may undergo auto-oxidation, cleavage at the ethylene oxide subunits and hydrolysis of the fatty acid ester bond caused by presence of oxygen, metal ions, peroxides or elevated temperature.

The lowest peptide adhesion is obtained when using 0.5 mg Kolliphor HS 15, this being the quantity of Kolliphor HS 15 selected in the final composition of the drug product.
Selection of the Antioxidant Agent

The presence of a radical scavenger with its antioxidant properties allows protecting NeoB from the radiolysis.

We considered for the development studies the gentisic acid and ascorbic acid, as antioxidants for use in radiopharmaceutical preparations. Tests were made in order to identify the lowest amount of the antioxidant able to exert the desired protective function, without interfering with the radiolabelling.

The radiolabelling has been tested, varying the amount of antioxidant agents and keeping constant other parameters, primarily to identify the most suitable antioxidant and the concentration that is not hampering the ⁶⁸Ga-incorporation into the DOTA-peptide. As shown in the table below, the gentisic acid was identified as the best antioxidant agent because it does not interfere in the ⁶⁸Ga incorporation above the 98% in HPLC. The amount of gentisic acid selected is 200 μg.

TABLE 8
Selection of Anti-oxidant agents

| | | | HPLC | |
|---|---|---|---|---|
| | | | free | HPLC |
| | Antioxidant | Activity | ⁶⁸Ga³⁺ | ⁶⁸Ga- |
| NeoB (μg) | agent (mg) | (MBq) | (%) | NeoB (%) |
| 50 | No | 1328.67 | t0h 1.74 | t0 92.70 |
| | antioxidant | | t2h 1.77 | [symbol] |
| 50 | No | 1253.56 | t0h 1.98 | t0h 93.03 |
| | antioxidant | | t2h 1.95 | [symbol] |
| 50 | Ascorbic acid | 1232.47 | [symbol] | [symbol] |
| | (0.1 mg) | | | |
| 50 | Ascorbic acid | 1243.20 | [symbol] | t0h 92.0 |
| | (0.5 mg) | | | |
| 50 | Gentisic acid | 962.00 | t0h 1.54 | t0h 94.13 |
| | (0.1 mg) | | t2h 1.51 | t2h 92.50 |
| 50 | Gentisic acid | 1134.79 | t0h 1.90 | t0h 94.00 |
| | (0.1 mg) | | t2h 1.88 | t2h 92.75 |
| 50 | Gentisic acid | 1288.71 | t0h 1.38 | t0h 94.06 |
| | (0.2 mg) | | t2h 1.54 | t2h 94.71 |
| | | | t4h 1.04 | t4h 94.86 |
| 50 | Cientisic acid | 1226.55 | t0h 1.75 | t0h 93.50 |
| | (0.2 mg) | | t2h 1.53 | t2h 94.84 |
| | | | t4h 1.69 | t4h 93.79 |
| 50 | Cientisic acid | 1082.25 | t0h 1.61 | t0h 95.12 |
| | (0.2 mg) | | t2h 1.63 | t2h 95.09 |
| | | | t4h 1.47 | t4h 95.07 |
| 50 | Gentisic acid | 1298.70 | t0h 1.62 | t0h 93.03 |
| | (0.35 mg) | | t4h 1.67 | t4h 92.95 |
| 50 | Gentisic acid | 1216.93 | t0h 1.88 | t0h 92.88 |
| | (0.35 mg) | | t4h 1.73 | t4h 92.63 |
| 50 | Gentisic acid | 1200.65 | t0h 1.97 | t0h 93.46 |
| | (0.5 mg) | | [symbol] | t2h 93.42 |
| 50 | Gentisic acid | 1144.04 | [symbol] | t0h 93.21 |
| | (0.5 mg) | | t2h 1.96 | t2h 93.43 |
| 50 | Gentisic acid | 625.30 | [symbol] | t0h 93.56 |
| | (1.0 mg) | | | |

Note:
Out of specification results are indicated in underlined bold font

Selection of the bulking agent

The formulation was finally completed with the addition of a bulking agent needed for the product freeze-drying process.

Among the bulking agents usually proposed for the lyophilisation of peptides, the drug product manufacturer tested inositol and mannitol.

TABLE 9
Selection of the Bulking agent tests

| | Antioxidant | | HPLC | HPLC |
|---|---|---|---|---|
| | agent | Activity | free ⁶⁸Ga³⁺ | ⁶⁸Gia-NeoB |
| NeoB (μg) | (mg) | (MBq) | (%) | (%) |
| 50 | Inositol | 1168.83 | 93.85 | 1.61 |
| | (20 mg) | | | |
| 50 | Inositol | 1216.56 | 92.99 | 1.98 |
| | (40 mg) | | | |
| 50 | Mannitol | 1141.45 | 94.06 | 1.84 |
| | (20 mg) | | | |
| 50 | Mannitol | 1203.98 | 93.59 | 1.87 |
| | (40 mg) | | | |

Mannitol was selected, as it is the most commonly used in the lyophilisates and it is known to produce a cake with good characteristics in terms of aspect, stability and moisture in freeze-drying processes. Moreover, mannitol is described in literature as good scavengers of OH radicals.

2.2.1.3 Radiolabelling Procedure

Based on the 2-vials design, a 3-step labelling procedure has been developed as follows:
1. direct reconstitution of the lyophilized formulation (Vial 1) with the solution of ⁶⁸Ga in HCl provided by the ⁶⁸Ge/⁶⁸Ga generator in the heating block (verify that the temperature has already reached 95° C. before starting elution).
2. addition of the necessary volume of Reaction buffer (Vial 2)
3. heating at 95° C. for at least 7 minutes (do not exceed 10 minutes heating)

At this point the ⁶⁸Ga-NeoB solution is ready for administration.

During the labeling procedure development, different time and temperature conditions have been tested.

The dependence of the labeling efficiency on the temperature has been studied to identify a value giving a good incorporation in a timeframe compatible with the short half-life of ⁶⁸Ga.

The incorporation of the ⁶⁸Ga into the DOTA chelating moiety is known to require heating to be accomplished.

The first tested labelling conditions were: labeling at 80, 85 and 95° C. with different reaction times (3, 5 and 7 minutes). These tests were performed using the following product formulation:
peptide (50 μg),
mannitol (20 mg),
gentisic acid (0.2 mg),
Kolliphor HS 15 (0.5 mg),
Hydroxy Propyl β Cyclodextrin (3 mg).

The formulation tested in these initial tests included the solubilizing agent, Hydroxy Propyl β Cyclodextrin. However, later on during development, similar tests were performed with the same formulation, but without Hydroxy Propyl β Cyclodextrin, obtaining good radiochemical and chemical purity. Additionally, the adhesion of peptide was also shown not to be affected by the absence of Hydroxy Propyl β Cyclodextrin, which was therefore not included in the final formulation. At 80° C. and 85° C. the radiometric analysis showed adequate incorporation in 7 minutes.

At 95° C., the incorporation is completed only after 7 minutes.

Based on these observations, 95° C. for 7 minutes showed to be the most conservative labelling condition, able to guarantee incorporation above 98% without significant fragmentation, even in the case of oscillation of the temperature in the range of ±15° C.

TABLE 10
Labelling at different temperatures and times

| | | HPLC | HPLC | ITLC |
|---|---|---|---|---|
| Labelling | Labelling | ⁶⁸Ga- | free | ⁶⁸Ga- |
| temperature | time | NeoB | ⁶⁸Ga³⁺ | NeoB |
| (° C.) | (min) | (%) | (%) | (%) |
| 80 | 7 | 94.8 | 1.59 | 98.4 |
| 85 | 7 | 94.8 | 1.33 | 99.2 |
| 95 | 3 | 94.2 | 2.3 | 98.5 |
| 95 | 5 | 93.8 | 2.2 | — |
| 95 | 7 | 93.8 | 1.6 | 99.3 |

Moreover, in order to increase the robustness of the labelling procedure, the addition of Reaction buffer (Vial 2) at room temperature (RT) was assessed (and only after addition of the Reaction buffer the labelling reaction was performed at 95° C.). Results showed in Table 11 confirm that good radiochemical purity is obtained also in these conditions.

TABLE 11
Elution and buffer adding at RT

| | | | HPLC | |
|---|---|---|---|---|
| | Labelling | | ⁶⁸Ga- | HPI.C |
| | temperature | Activity | NeoB | free ⁶⁸Ga³⁺ |
| NeoB (μg) | (° C.) | (MBq) | (%) | (%) |
| 50 | 95 | 1226.55 | t0h 93.50 | t0h 1.75 |

### 2.2.1.4 Final Selected Formulation (Vial 1)

Based on all above mentioned development studies the final composition of the NeoB 50 μg, powder for solution for injection (Vial 1) is as follows:

TABLE 12
Final composition of Vial l powder for solution for injection

| | Composition | | |
|---|---|---|---|
| Component | (per vial) | | Function |
| NeoB | 50 | μg | Drug Substance |
| Gentisic acid | 200 | μg | Radiolysis protector/Antioxidant |
| Mannitol | 20 | mg | Bulking agent |
| Macrogol 15 | 500 | μg | Tensioactive agent |

hydroxystearate
(Kolliphor HS15)

The final formulation has been tested in regard of the radiolabelled product in order to confirm the results obtained during the development.

TABLE 13
Radiolabelling tests performed with the final formulation

| | | | ITLC | HPLC | HPLC | |
|---|---|---|---|---|---|---|
| | | | ⁶⁸Ga- | free | ⁶⁸Ga- | Peptide |
| | Activity | | NeoB | ⁶⁸Ga³⁺ | NeoB | Adhesion |
| | (MBq) | pH | (%) | (%) | (%) | (%) |
| NeoB (50 μg) | 1091.5 | 3.6 | 98.9 | 1.6 | 93.8 | 5.8 |
| Mannitol (20 mg) | | | | | | |
| Kolliphor HS15 (500 μg) | 913.9 | 3.5 | 99.1 | 1.5 | 93.3 | 4.8 |
| Gentisic acid (200 μg) | 836.2 | 3.8 | 99.7 | 1.4 | 94.9 | 5.0 |

As shown in the Table 13, good radiochemical purity results by both ITLC and HPLC (>92%) were obtained after three independent radiolabelling tests performed with the final formulation. It is also important to note that free gallium (by HPLC) was always below 2%. Finally, the peptide adhesion to the glass was also tested during these radiolabelling tests, confirming that presence of Kolliphor HS15 is necessary to maintain the peptide adhesion at acceptable levels.

2.2.1.5 Quality Specification Evaluation

In order to correctly define the quality specification, a set of preliminary experiments were performed, as summarized below.

Labelling pH

The labelling pH is one of the crucial parameters to obtain good results in terms of radiolabelling yield of DOTA-peptides with ⁶⁸GaCl₃ due to its particular chemical behavior. In order to define a pH range within which the labelling provides good results the NeoB formulation labelled with ⁶⁸Gallium was tested maintaining the range of pH between 3.0-4.0. The labelling has been tested, varying the volume of reaction buffer added and keeping constant other parameters. As shown in Table 14 and in Table 15, pH variations within the range 3.0-4.0 do not affect the success of the labelling. The radiolabelled product obtained meets the radiochemical purity specification.

TABLE 14
Tests performed with the final formulation at lower pH

| | | | ITLC | HPLC | HPLC |
|---|---|---|---|---|---|
| | | | ⁶⁸Ga- | free | ⁶⁸Ga- |
| | Activity | | NeoB | ⁶⁸Ga³⁺ | NeoB |
| Formulation | (MBq) | pH | (%) | (%) | (%) |
| NeoB (50 μg) | 621.6 | 3.01 | 100.00 | 1.64 | 94.59 |
| Mannitol (20 mg) | 658.6 | 3.05 | 100.00 | 1.78 | 94.37 |
| Kolliphor HS15 | | | | | |
| (500 μg) | | | | | |
| Gentisic acid | | | | | |
| (200 μg) | | | | | |

TABLE 15
Tests performed with the final formulation at higher pH

| | | | ITLC | HPLC | HPLC |
|---|---|---|---|---|---|
| | | | ⁶⁸Ga- | free | ⁶⁸Ga- |
| | Activity | | NeoB | ⁶⁸Ga³⁺ | NeoB |
| Formulation | (MBq) | pH | (%) | (%) | (%) |
| NeoB (50 μg) | 721.5 | 3.82 | 99.40 | 1.98 | 93.20 |
| Mannitol (20 mg) | 603.1 | 4.03 | 99.03 | 1.95 | 94.78 |
| Kolliphor HS15 (500 | | | | | |
| μg) | | | | | |
| Gentisic acid (200 μg) | | | | | |

Gentisic Acid Vs Volumic Activity

The tests were performed in order to evaluate the effect of gentisic acid as radiolytic scavengers when the labelling is performed with the highest volumic activity of ⁶⁸GaCl₃ that the ⁶⁸Ge/⁶⁸Ga generator can provide at that time. In order to have the highest possible volumic activity, a fractionated elution was performed; only the portion with the highest activity was used for the labelling.

The protective effect was verified by monitoring the peptide fragmentation over time in presence of different amount of gentisic acid (0.20 mg and 0.35 mg). The results (see Table 16) confirmed almost the same positive effects for both tests. Therefore, the lowest amount of gentisic acid (200 μg) sufficient to achieve good level of protection from radiolysis was chosen.

TABLE 16
Radiolabelling tests performed with the highest volumic activity
(fractionated elution)

| | | | | | ITLC |
|---|---|---|---|---|---|
| | | HPLC | HPLC | ITLC | non- |
| Gentisic | Volumic | ⁶⁸Ga- | free | ⁶⁸Ga- | complexed |
| acid | activity | NeoB | ⁶⁸Ga³⁺ | NeoB | ⁶⁸Ga³⁺ |
| (mg) | (MBq/ml) | (%) | (%) | (%) | (%) |
| 0.35 | 402.19 | t0h 95.66 | t0h 0.95 | t0h 100% | 0.0% |
| | | t2h 95.90 | t2h 0.86 | | |
| 0.20 | 376.66 | t0h 94.75 | t0h 1.30 | t0h 100% | 0.0% |
| | | t2h 95.98 | t2h 0.98 | | |

Additionally, in order to test if lower amounts of gentisic acid are still able to act as antioxidant agent in the final formulation, an initial test was performed using 0.1 mg of gentisic acid. The results of the radiolabelling test performed in these conditions are shown in Table 17, and confirm that even in the presence of lower amount of gentisic acid good radiochemical purity can be obtained. Nevertheless, in order to guarantee that good radiochemical purity is obtained with a higher activity of the generator, the amount of gentisic acid in the final formulation has been conservatively kept at 200 μg.

TABLE 17
Radiolabelling with higher concentration of ⁶⁸Ga and lower
concentration of Gentisic acid

| | | ITLC | HPLC | HPLC |
|---|---|---|---|---|
| | | ⁶⁸Ga- | free | ⁶⁸Ga- |
| | Activity | NeoB | ⁶⁸Ga³⁺ | NeoB |
| Formulation | (MBq) | (%) | (%) | (%) |
| NeoB1 (50 μg) | 962.0 | 99.1 | 1.54 | 94.13 |
| Mannitol (20 mg) | | | | |
| Kolliphor HS15 (500 μg) | | | | |
| Gentisic acid (100 μg) | | | | |

Scale-Up Batch—Tests Results of the ⁶⁸Ga Radiolabelled Product

Table 1Table 18 summarizes the two radiolabelling tests performed with the scale-up batch NeoB Vial 1. The results showed that the radiolabelled drug product ⁶⁸Ga-NeoB obtained with the scale-up batch of Vial 1 meets the radiochemical purity specifications for up to 4 hours after the end of radiolabelling reaction.

TABLE 18
Radiolabelling tests performed with the Scale-up
Batch (CT005 16001)

| | | ITLC | HPLC | HPLC |
|---|---|---|---|---|
| Activity | | ⁶⁸Ga-NeoB | free⁶⁸Ga³⁺ | ⁶⁸Ga-NeoB |
| (MBq) | pH | (%) | (%) | (%) |
| 588.3 | 3.9 | 99.50 | t0h 0.61 | t0h 97.23 |
| | | | t2h 0.59 | t2h 97.69 |
| | | | t4h 0.49 | t4h 97.79 |
| 592.0 | 3.8 | 99.42 | t0h 0.70 | t0h 96.72 |
| | | | t2h 0.55 | t2h 97.50 |
| | | | t4h 0.60 | t4h 97.42 |

### REFERENCES

1. Sah B R, Burger I A, Schibli R, Friebe M, Dinkelborg L, Graham K, Borkowski S, Bacher-Stier C, Valencia R, Srinivasan A et al: Dosimetry and First Clinical Evaluation of the New 18F-Radiolabeled Bombesin Analogue BAY 864367 in Patients with Prostate Cancer. J Nucl Med 2015, 56(3):372-378.
2. Kahkonen E, Jambor I, Kemppainen J, Lehtio K, Gronroos T J, Kuisma A, Luoto P, Sipila H J, Tolvanen T, Alanen K et al: In vivo imaging of prostate cancer using [68Ga]-labeled bombesin analog BAY86-7548. Clin Cancer Res 2013, 19(19):5434-5443.
3. Maina T, Bergsma H, Kulkarni H R, Mueller D, Charalambidis D, Krenning E P, Nock B A, de Jong M, Baum R P: Preclinical and first clinical experience with the gastrin-releasing peptide receptor-antagonist [(68)Ga]SB3 and PET/CT. Eur J Nucl Med Mol Imaging 2016, 43(5):964-973.
4. Dimitrakopoulou-Strauss A, Hohenberger P, Haberkorn U, Macke H R, Eisenhut M, Strauss L G: 68Ga-labeled bombesin studies in patients with gastrointestinal stromal tumors: comparison with 18F-FDG. J Nucl Med 2007, 48(8):1245-1250.
5. Velikyan I, Xu H, Nair M, Hall H: Robust labeling and comparative preclinical characterization of DOTA-TOC and DOTA-TATE. Nucl Med Biol 2012, 39(5):628-639.

1. A method for labeling a gastrin-releasing peptide receptor (GRPR) antagonist with a radioactive isotope, said method comprising the steps of:
i. providing a first vial comprising said GRPR antagonist in dried form,
ii. adding a solution of said radioactive isotope into said first vial, thereby obtaining a solution of said GRPR antagonist with said radioactive isotope,
iii. mixing the solution obtained in ii. with at least a buffering agent and incubating it for a sufficient period of time for obtaining said GRPR antagonist labeled with said radioactive isotope, and,
iv. optionally, adjusting the pH of the solution.

2. The method of claim 1, wherein the first vial at step i. is a reaction vial comprising said GRPR antagonist and a buffering agent.

3. The method of claim 1, wherein step iii, comprises mixing the solution obtained in ii. with at least a reaction solution comprising a buffering agent and incubating it for a sufficient period of time for obtaining said GRPR antagonist labeled with said radioactive isotope.

4. The method of claim 1, wherein the GRPR antagonist is a compound of formula (I):

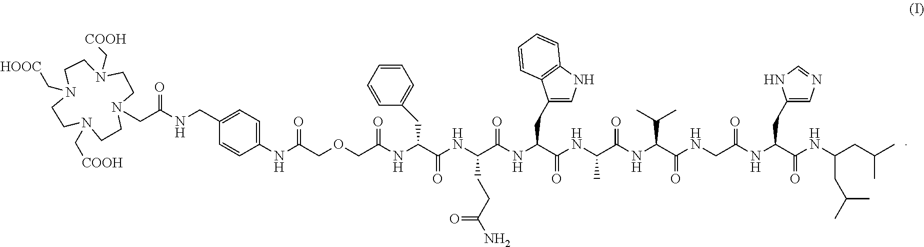 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

5. The method of claim 1, wherein said GRPR antagonist is comprised in said first vial at an amount between 20 and 60 μg.

6. The method of claim 1, wherein said first vial further comprises gentisic acid as a radiolysis protector.

7. The method of claim 1, wherein said first vial further comprises mannitol.

8. The method of claim 1, wherein said first vial further comprises macrogol 15 hydroxystearate.

9. (canceled)

10. The method of claim 1, wherein the GRPR antagonist is a compound of Formula
(I), 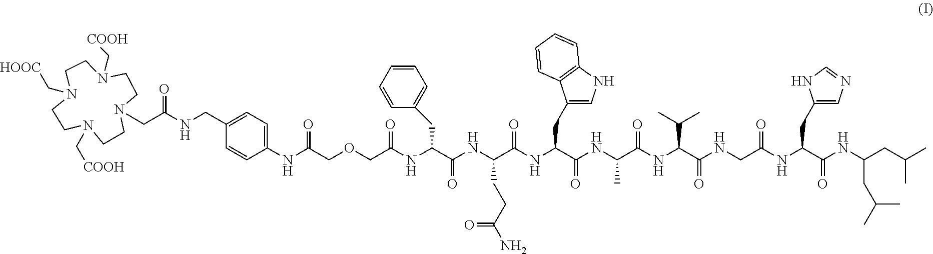 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]
and the radioactive isotope is ⁶⁸Ga.

11. A powder for solution for injection, comprising the following components in dried forms:
i. A GRPR antagonist of the following formula:
C—S—P
wherein:
C is a chelator capable of chelating a radioactive isotope;
S is an optional spacer covalently linked between C and the N-terminal of P;
P is a GRPR peptide antagonist of the general formula:
Xaa1-Xaa2-Xaa3-Xaa4Xaa5-Xaa6-Xaa7-Z; wherein
Xaa1 is not present or is selected from the group consisting of amino acid residues Asn, Thr, Phe, 3-(2-thienyl) alanine (Thi), 4-chlorophenylalanine (Cpa), α-naphthylalanine (α-Nal), β-naphthylalanine (β-Nal), 1,2,3,4-tetrahydronorharman-3-carboxylic acid (Tpi), Tyr, 3-iodo-tyrosine (0-I-Tyr), Trp and pentafluorophenylalanine (5-F-Phe) (all as L- or D-isomers);
Xaa2 is Gln, Asn or His;
Xaa3 is Trp or 1, 2, 3, 4-tetrahydronorharman-3-carboxylic acid (Tpi);
Xaa4 is Ala, Ser or Val;
Xaa5 is Val, Ser or Thr;
Xaa6 is Gly, sarcosine (Sar), D-Ala, or β-Ala;
Xaa7 is His or (3-methyl)histidine (3-Me)His;
Z is selected from —NHOH, —NHNH2, —NH-alkyl, —N(alkyl)2, and —O-alkyl or Z is 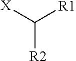
wherein X is NH (amide) or O (ester) and R1 and R2 are the same or different and selected from a proton, an optionally substituted alkyl, an optionally substituted alkyl ether, an aryl, an aryl ether or an alkyl-, halogen, hydroxyl, hydroxyalkyl, amine, amino, amido, or amide substituted aryl or heteroaryl group;
ii. a radiolysis protector;
iii. a bulking agent; and,
iv. optionally a surfactant.

12. The powder for solution for injection according to claim 11, wherein said GRPR antagonist is a compound of Formula (I):

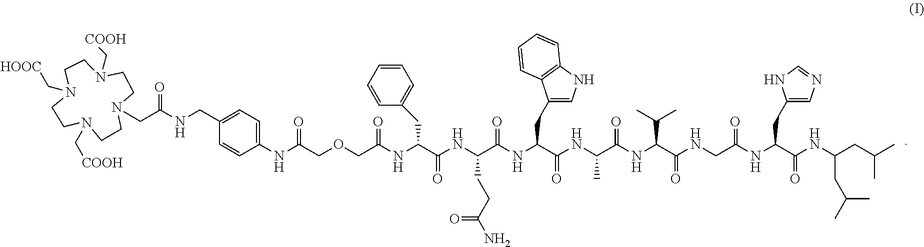 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]

13. The powder for solution for injection of claim 11, comprising the following components:
a GRPR peptide antagonist of formula (I) at an amount between 20 and 60 μg; 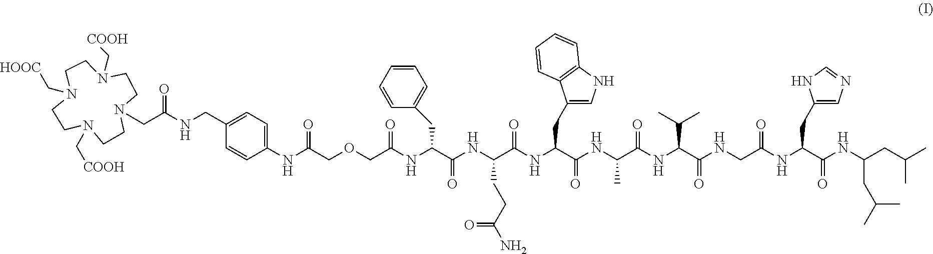 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]
gentisic acid at an amount between 50 and 250 μg;
mannitol at an amount between 10 and 30 mg; and
macrogol 15 hydroxystearate at an amount between 250 and 750 μg.

14. A kit comprising
i. a first vial with the following components in dried forms
a compound of formula (I): 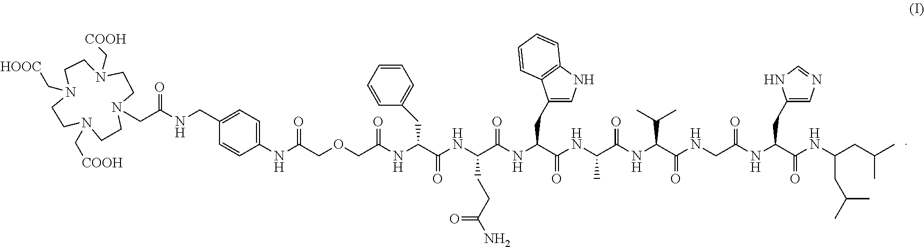 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]
a radiolysis protector,
optionally a bulking agent, and,
optionally a surfactant;
ii. a second vial comprising at least a buffering agent; and
iii. optionally, an accessory cartridge for eluting a radioactive isotope generated by a radioactive isotope generator.

15. A kit comprising
i. a single vial with the following components in dried forms
a compound of formula (I): 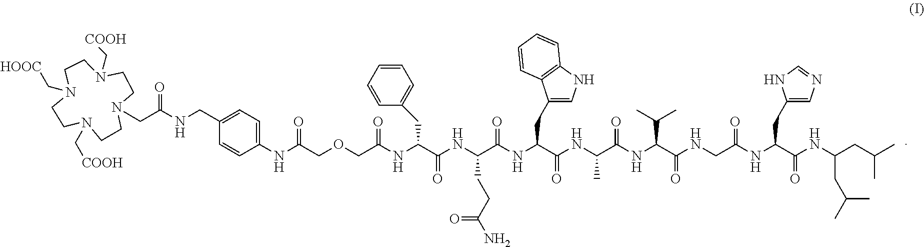 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]
a radiolysis protector,
optionally a bulking agent,
optionally a surfactant, and
at least a buffering agent, preferably in dried form; and
ii. optionally, an accessory cartridge for eluting a radioactive isotope generated by a radioactive isotope generator.

16. The kit of claim 14, wherein said first vial comprises the following components:
the compound of formula (I) at an amount between 20 and 60 μg; 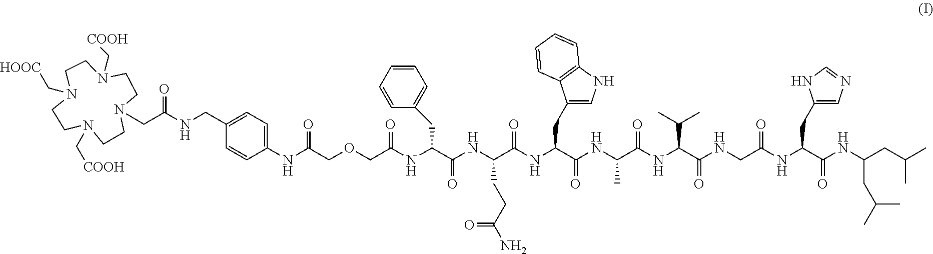 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)CN2CCN(CC(=O)O)CCN(CC(=O)O)CCN(CC(=O)O)CC2)cc1)C(C)C]
gentisic acid at an amount between 50 and 250 μg,
mannitol at an amount between 10 and 30 mg, and
optionally, macrogol 15 hydroxystearate at an amount between 250 and 750 μg.

17. The kit of claim 14, wherein all components of said first, vial are in dried forms.
